(19) United States
(12) Patent Application Publication
(10) Pub. No.: US 2025/0352331 A1
(43) Pub. Date: Nov. 20, 2025

(54) PROSTHETIC VALVES AND OVAL STENTS FOR FLOW BALANCING

(71) Applicant: EDWARDS LIFESCIENCES CORPORATION, Irvine, CA (US)

(72) Inventors: Maya Katz, Tel Aviv (IL); Benjamin Adam Watts, Or Akiva (IL); Sagi Libovitch, Sdot Yam (IL); Roei Mayo, Irvine, CA (US); Yulia Mordukhaev, Ramat Gan (IL)

(21) Appl. No.: 19/281,230

(22) Filed: Jul. 25, 2025

Related U.S. Application Data
(63) Continuation of application No. PCT/US2024/013459, filed on Jan. 30, 2024.
(60) Provisional application No. 63/482,177, filed on Jan. 30, 2023.

(51) Int. Cl. A61F 2/24; A61F 2/48

(52) U.S. Cl. CPC A61F 2/2418; A61F 2/482; A61F 2230/0008

(57) ABSTRACT

Systems and methods are described for modulating blood flow through a blood vessel. The systems may include one or more implantable devices including a stent frame positionable within the blood vessel, the stent frame having a substantially oval cross-sectional shape, and a valve defining an inflow end and an outflow end. The inflow end may be coupled to the stent frame and the outflow end defining an aperture and the valve may include at least one leaflet at the outflow end. The at least one leaflet may be positionable to overlay at least a portion of the aperture and configured to move in a radial direction that is perpendicular to a central axis of the stent frame.

### CROSS-REFERENCE TO RELATED APPLICATIONS

This application is a continuation of International Application No. PCT/US24/13459 filed Jan. 30, 2024, which claims the priority benefit of U.S. Provisional Patent Application Ser. No. 63/482,177, filed Jan. 30, 2023, the contents of each of which are herein incorporated by reference in their entireties.

### BACKGROUND

This disclosure relates generally to the field of medical devices and procedures, and more specifically to the field of blood flow management in blood vessels.

Patients that suffer from congestive heart failure (CHF) frequently also experience impaired renal function. Impaired renal function can be caused by increased systemic venous congestion resulting from low cardiac output and low blood pressure. In such patients, the renal pressure gradient (i.e., between the renal arteries and renal veins) may be decreased due to elevated renal venous pressure, which may also lower a rate at which the kidney filters blood (e.g., the glomerular filtration rate (GFR)). A low GFR may be indicative of chronic kidney disease (CKD) that can eventually lead to end stage renal failure in the patient.

### SUMMARY

Described herein are one or more methods and/or devices to facilitate management of blood flow through and/or into one or more blood vessels associated with the renal arteries and/or renal veins. There is a need for new and useful systems and methods that improve GFR and reduce blood volume retention in patients with congestive heart failure (CHF) and/or chronic kidney disease (CKD).

In some aspects, the techniques described herein relate to an implantable device for modulating blood flow through a blood vessel, the implantable device including: a stent frame positionable within the blood vessel, the stent frame having a substantially oval cross-sectional shape; and a valve defining an inflow end and an outflow end, the inflow end being coupled to the stent frame and the outflow end defining an aperture, the valve including at least one leaflet at the outflow end, the at least one leaflet being positionable to overlay at least a portion of the aperture and configured to move in a radial direction that is perpendicular to a central axis of the stent frame, wherein the valve is configured to cause the at least one leaflet to move in the radial direction to reduce a cross-sectional area of the aperture and restrict flow through the valve in response to elevated pressure within the blood vessel.

In some aspects, the techniques described herein relate to an implantable device for modulating blood flow through a blood vessel, the device including: a stent frame positionable within the blood vessel, the stent frame having a substantially oval cross-sectional shape; a valve defining an inflow end and an outflow end defining an aperture, the valve including at least one leaflet at the outflow end, the at least one leaflet being positionable to overlay at least a portion of the aperture and configured to move in a radial direction that is perpendicular to a central axis of the stent frame; a first elastomeric member having a first opening and a second opening and defining a first lumen, the first opening being coupled to a portion of the stent frame, the second opening being coupled to a portion of the inflow end of the valve; and a second elastomeric member having a third opening and a fourth opening and defining a second lumen, the third opening being coupled to a portion of the stent frame, the fourth opening being coupled to a portion of the inflow end of the valve, wherein the valve is configured to cause the at least one leaflet to move in the radial direction to reduce a cross-sectional area of the aperture and restrict flow through the valve in response to elevated pressure within the blood vessel.

In some aspects, the techniques described herein relate to a method of modulating a pressure gradient between a first kidney and a second kidney of a subject, the method including: introducing an implantable device in a renal vein, the device including: a stent frame with an oval-shaped cross-sectional area; and a valve defining an inflow end and an outflow end, the inflow end being coupled to the stent frame and the outflow end defining an aperture, the valve including at least one leaflet at the outflow end of the valve, the at least one leaflet being positionable to overlay at least a portion of the aperture and configured to move in a radial direction that is perpendicular to a central axis of the stent frame; and actuating the implantable device to modulate a flow of blood within the renal vein, the actuating including causing the at least one leaflet to move in the radial direction to increase or decrease a cross-sectional area of the aperture.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates a side view of an example flow modulating device implantable in a renal vein to modulate blood flow through the renal vein.

FIG. 2A illustrates a side perspective view of an example non-circular stent frame for use with the flow modulating devices described herein.

FIG. 2B illustrates an example top down view of an example non-circular stent frame for use with the flow modulating devices described herein.

FIG. 2C illustrates another example top down view of an example non-circular stent frame for use with the flow modulating devices described herein.

FIG. 2D illustrates a top down view of an example circular stent frame for use with the flow modulating devices described herein.

FIG. 2E illustrates an axial view of an example non-circular stent frame for use with the flow modulating devices described herein.

FIGS. 3A-3C illustrate example flow modulating devices in various stages of operation.

FIG. 4A illustrates a side perspective view of an example valve for use with the flow modulating devices described herein.

FIG. 4B illustrates a top down view of an example valve for use with the flow modulating devices described herein.

FIG. 4C illustrates a top down view of an example valve for use with the flow modulating devices described herein.

FIG. 4D illustrates a top down view of an example valve for use with the flow modulating devices described herein.

FIG. 4E illustrates a top down view of an example valve for use with the flow modulating devices described herein.

FIG. 4F illustrates a top down view of an example valve for use with the flow modulating devices described herein.

FIG. 5 illustrates a side view of two example flow modulating devices implanted in a left renal vein and a right renal vein, respectively.

FIG. 6 illustrates a side view of an example flow modulating device implanted in a right renal vein.

FIG. 7 illustrates a side view of an example flow modulating device implanted in a right renal vein.

FIG. 8 illustrates a side view of an example flow modulating device implanted in a right renal vein.

FIG. 9 illustrates a side view of example flow modulating devices implanted in a left renal vein and a right renal vein and a portion of an inferior vena cava.

FIG. 10 illustrates a block diagram of an example system for modulating blood flow through one or more blood vessels.

FIG. 11 illustrates a flow diagram of an example process for improving a pressure gradient across the kidneys.

FIG. 12 illustrates a schematic representation of portions of a human subject in which the devices described herein may be implanted.

FIGS. 13A and 13B illustrate a side view and a top down view of an example flow modulating device for implantation into portions of an IVC, a left renal vein, and/or a right renal vein.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating and/or balancing of blood flow through a blood vessel, such as one or more renal veins. The modulating and/or balancing of blood flow may be performed by the prosthetic devices described herein to occlude, partially occlude, and/or otherwise modulate or regulate blood flow to or through a portion of a blood vessel. In some examples, such management of blood flow to or through a blood vessel may result in additionally modulating pressure across one or both kidneys and/or other organs of the body. That is, the devices described herein may be used to manage blood flow to reduce central venous pressure. In particular, the devices described herein may be implanted in a section of vessel between the Inferior Vena Cava (i.e., IVC) and a renal outlet (e.g., a renal vein). Such devices may be actuated to reduce renal outlet pressure and thus improve ureter output and/or kidney function by increasing a gradient across one or both kidneys.

The examples presented herein may relate to providing devices, methods, and/or methods of treatment (MOTs) for modulating, regulating and/or otherwise managing blood flow to or through one or more blood vessels. In some examples, the devices described herein may include blood flow management devices for reducing blood flow through a blood vessel, such as the inferior vena cava or related vessels. The terminology of restricting blood flow, regulating blood flow, modulating blood flow, managing blood flow, and balancing blood flow cause regulation of blood pressure, modulation of blood pressure, management of blood pressure, and/or balancing of blood pressure. As such, for example, a flow modulation device is synonymous with a pressure regulating device (i.e., a flow regulator is synonymous with a pressure regulator).

Managing blood flow through the IVC can be achieved by the devices described herein to provide an advantage of increasing blood volume passing through the kidneys. In particular, the devices described herein may improve a pressure gradient across the kidneys by decreasing central venous pressure by modulating, balancing, or otherwise modifying blood flow through the IVC and/or one or more renal veins, resulting in improved kidney blood flow and function.

In some examples, the devices, methods, and/or MOTs described herein may be utilized to solve a technical problem of unwanted pressure increases in the IVC and/or renal veins in patients that have chronic kidney disease (CKD) and/or congestive heart failure (CHF). For example, patients with CKD and/or CHF may exhibit reduced kidney function when a pressure gradient across the kidneys is low because of venous congestion or the like. The devices, methods, and/or MOTs described herein can improve the pressure gradient across the kidneys in a manner that also improves GFR and reduces blood volume retention.

In some examples, the devices, methods, and/or MOTs described herein may also be configured to limit central venous volume to operate in a bi-modal fashion. For example, the devices described herein may be configured to reduce venous pressure when the patient is at rest, yet allow undisturbed or minimally disturbed venous flow when the patient exercises, to meet the dynamic blood flow and/or pressure requirements of the renal system.

In some examples, the devices described herein may be configured to reduce a back flow of blood toward a kidney from a renal vein by at least partially obstructing a portion of the renal vein. The partial or full obstruction may be triggered in response to detection of a pressure (e.g., using a sensor) within an IVC that is greater than a predefined pressure threshold (e.g., pressure level) for the IVC. For example, the predefined pressure threshold may be between about 10 mmHG and about 25 mmHG; between about 10 mmHG and about 12 mm HG; between about 12 mmHG and about 15 mmHG; between about 15 mmHG and about 20 mmHG; between about 20 mmHG and about 25 mmHG. In some examples, the partial or full obstruction may be triggered in response to detection of a pressure (e.g., using a sensor) within a renal vein that is greater than a predefined pressure threshold for the renal vein. Such predefined pressure thresholds may be used as a basis to determine whether a patient is exhibiting low vessel pressure (e.g., below the predefined pressure threshold) or high vessel pressure (e.g., above the predefined pressure threshold). When vessel pressure is determined to be high, the devices, methods, and/or MOTs can provide a technical solution to the technical problem recited above. For example, each of the devices described herein may be used to decrease pressure within one or more vessels to improve outflow from one or both kidneys to the IVC thereby promoting improved function of one or both kidneys. In particular, the devices, methods, and/or MOTs described herein can be used to increase or maintain a renal pressure gradient in CKD patients suffering from venous congestion, which provides a technical effect of enabling the kidneys to more effectively filter blood.

The examples described herein can perform blood flow management actively and/or passively to assist in increasing or maintaining a renal pressure gradient even when a surge in blood volume occurs in one or more vessels of the venous system.

### Systems and Devices

Disclosed herein are systems and methods for modulating blood flow through a blood vessel. In some examples, the implantable flow modulating devices described herein may be used in blood flow occlusion therapy. For example, the devices described herein may relate to venous occlusion therapy using implantable and/or electronically controlled flow modulating devices for the treatment of congestive heart failure. Some devices may be non-implantable or partially implantable. In some examples, the devices described herein generally function to occlude or partially occlude a blood vessel, such one or more renal veins and/or partially occlude the IVC. In some examples, the devices described herein have been contemplated for use in a patient/user having chronic or congestive heart failure and/or chronic kidney disease, but may be used in any vessel that would benefit from flow modulation therethrough.

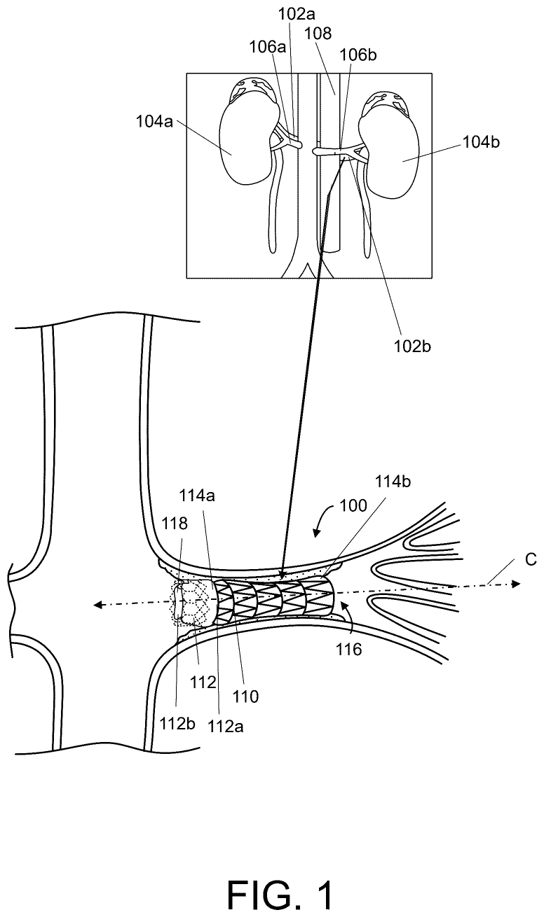

FIG. 1 illustrates a side view of an example flow modulating device 100. The device 100 may be an implant that is implantable in a blood vessel to modulate blood flow through the blood vessel. The device 100 may represent a transcatheter-deliverable device configured to operate in a manner that minimizes risk of thrombosis. The device 100 may be implantable in a renal vein 102a of right kidney 104a and/or renal vein 102b of left kidney 104b to modulate blood flow through the respective renal vein(s). The device 100 may function to modulate a volume of blood flowing from the kidney to the inferior vena cava to reduce and/or otherwise modulate venous pressure.

In general, the renal veins 102a, 102b branch from an IVC 108 and into the respective kidney 104a, 104b. In particular, the renal veins 102a, 102b fluidly connect the IVC 108 to a respective kidney. High venous pressure in the renal veins can result from venous congestion in one or more portions of the renal veins 102a, 102b. High venous pressure can reduce the pressure gradient between the renal arteries 106a, 106b and the renal veins 102a, 102b, which in turn can lower the rate at which the kidney filters blood (i.e., GFR). The device 100 may be implanted in one or both renal veins 102a, 102b to improve flow from one or both kidneys 104a, 104b to the IVC 108. In addition, device 100 may be implanted in one or both renal veins 102a, 102b to improve GFR function and/or to reduce blood volume retention in the veins.

As shown in FIG. 1, the device 100 includes a stent frame 110 and a valve 112. The stent frame 110 may be coupled to the valve 112 and the resulting device may be positionable within a blood vessel. The stent frame 110 may be a self-expanding or balloon expandable frame that may be delivered into the blood vessel (e.g., via femoral access, jugular access, subclavian access, or transfemoral access) using a sheathed catheter (not shown).

In general, the stent frame 110 may be an elongated tubular member having a first end 114a with a first opening (covered by valve 112). The stent frame 110 may further include a second end 114b with a second opening 116, a lumen extending between the first opening and the second opening, and/or a stent length extending between the first end 114a and the second end 114b. In some examples, the stent frame 110 may have a non-circular cross-sectional shape. For example, the stent frame 110 may be substantially tubular-shaped with a substantially oval (e.g., elliptical) cross-sectional shape about a central axis (C). In some examples, the stent frame 110 may have a substantially circular cross-sectional shape. For example, the stent frame 110 may be substantially tubular-shaped with a substantially circular cross-sectional shape about the central axis (C). The cross-sectional shape of the stent frame 110 may further be any shape, including a triangle, a peanut, a figure-8, and/or a kidney shape.

The stent frame 110 may be configured to receive the valve 112. For example, the stent frame 110 may be coupled to the valve 112. In some examples, the stent frame 110 may be removably coupled to the valve 112. In some examples, the stent frame 110 may be coupled to the valve by one or more elastomeric members representing at least one tubular structure coupled to both the stent frame 110 and the valve 112, as discussed in detail throughout this disclosure.

The description of the stent frame 110 may be understood to relate to, and/or describe aspects of, any of the stent frames described herein; that is, description of aspects of any example stent frame of the present disclosure may be understood to be implementable in any other example stent frame of the present disclosure.

The valve 112 can be operated to improve a pressure gradient across the kidneys 104a, 104b by protecting blood outflow from the kidneys from high central venous pressures. For example, the valve may be a one-way valve configured to modulate blood flow through the valve 112 and from at least one organ (e.g., a kidney). In operation, the valve 112 may be triggered to close in response to pressure detected within the IVC (or a related vein) being greater than a predefined threshold pressure. In some examples, the valve 112 may be configured to restrict blood flow through an aperture 118 of the valve 112 in response to an elevated pressure within the blood vessel in which the device 100 is implanted.

In some examples, the valve 112 may have a non-circular cross-sectional shape. For example, the valve 112 may be substantially tubular-shaped with a substantially oval cross-sectional shape about the central axis (C). In some examples, the valve 112 may have a substantially circular cross-sectional shape. For example, the valve 112 may be substantially tubular-shaped with a substantially circular cross-sectional shape about the central axis (C).

The valve 112 may define an inflow end 112a and an outflow end 112b. The inflow end 112a may be coupled to the stent frame 110 at first end 114a. The outflow end 112b may define the aperture 118 that may be configured to open or partially open (e.g., or partially close) to modulate blood flow therethrough. For example, device 100 may be triggered to reduce a cross-sectional area of the aperture 118 to restrict flow through the valve in response to elevated pressure within the blood vessel.

The apertures described herein (e.g., aperture 118, aperture 410, aperture 518, aperture 528) may be substantially oval or substantially circular and may have a diameter of about 5 millimeters to about 20 millimeters. For example, the apertures described herein may have a diameter of about 5 millimeters to about 10 millimeters; about 10 millimeters to about 15 millimeters; or about 15 millimeters to about 20 millimeters.

In some examples, the valve 112 includes at least one leaflet (not shown) at the outflow end 112b. The at least one leaflet may be positionable to overlay at least a portion of the aperture 118. The at least one leaflet may be configured to move in a radial direction that is perpendicular to the central axis (C) of the stent frame 110. In some examples, the valve 112 is configured to cause the at least one leaflet to move in the radial direction to reduce a cross-sectional area of the aperture 118 and restrict flow through the valve 112 in response to elevated pressure within the blood vessel (e.g., renal vein 102a).

In some examples, the leaflet(s) described herein may be configured to reduce a back flow of blood toward one or both kidneys via a respective renal vein by partially or fully obstructing an aperture of the valve responsive to detecting a pressure within the IVC that is greater than a predefined pressure threshold.

The description of the valve 112 may be understood to relate to, and/or describe aspects of, any of the valves described herein; that is, description of aspects of any example valve of the present disclosure may be understood to be implementable in any other example valve of the present disclosure.

In some examples, the device 100 may be implanted in one or both renal veins 102a, 102b to provide an advantage of reducing back flow of blood toward the kidney(s) 104a, 104b via the respective renal vein(s) 102a, 102b. For example, the valve 112 may be caused to close or partially close in response to detecting blood flowing back into the renal vein 102a or renal vein 102b. The device 100 may further include controls, components, and/or other features that may actuate device 100 to modulate blood flow, as will be described in detail throughout this disclosure.

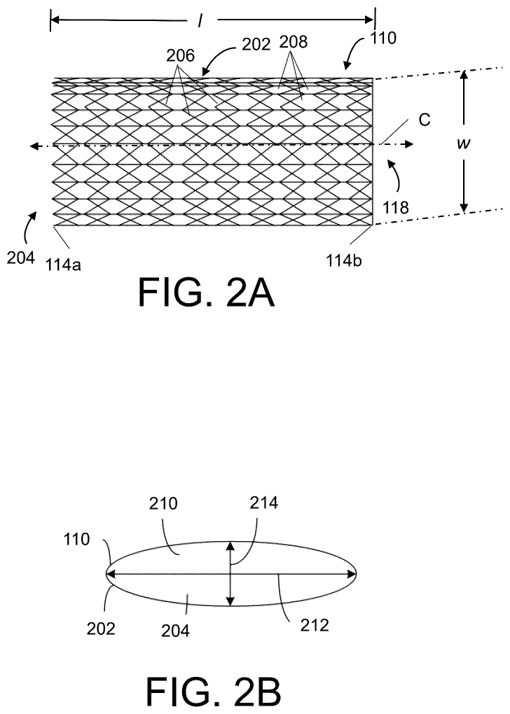

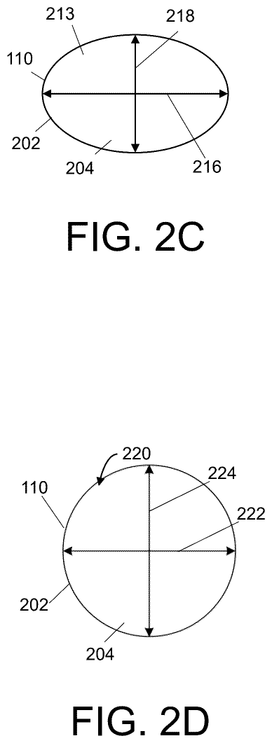

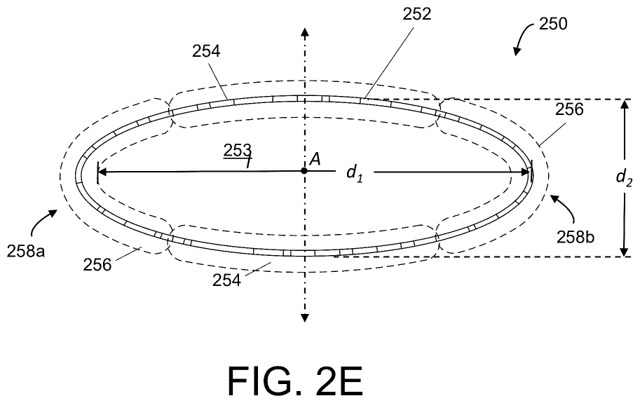

FIG. 2A illustrates a side perspective view of an example non-circular stent frame 110 for use with the flow modulating devices described herein. The stent frame 110 may include a substantially tubular stent wall 202 defining the first end 114a with a first opening 204, the second end 114b with the second opening (e.g., aperture 118), and a lumen extending between the first opening 204 and the second opening (e.g., aperture 118), and/or a stent length extending between the first end 114a and the second end 114b.

The stent wall 202 may be at least partially composed of a plurality of struts 206 and/or stent openings/cells 208 between the struts 206. The dimensions and/or shape of the stent frame 110 may vary based on the particular application and/or target implantation anatomy. For example, a stent length/may be selected to extend over all or a portion of an identified portion of a target blood vessel. The portion of the target blood vessel may be a non-compliant portion in which the device 100 may be implanted to ensure compliance of the blood vessel.

The stent frame 110 may have a length between about 30 millimeters to about 70 millimeters. In some examples, the stent frame 110 may have a length between about 30 millimeters to about 40 millimeters; about 40 millimeters to about 50 millimeters; about 50 millimeters to about 60 millimeters; or about 60 millimeters to about 70 millimeters. However, other sizes and/or shapes are also within the scope of this disclosure. In some examples, the stent frame 110 may represent about 80 percent of the length of the device 100. Put another way, the valve length ratio to device 100 length may be about 1:5.

The stent frame 110 may have a first diameter between about 5 millimeters and about 20 millimeters. In some examples, cross-section of the stent frame 110 is oval in shape. Accordingly, the first diameter (e.g., associated with a major axis of the stent) may pertain to a largest distance across the cross-section of the oval (i.e., longest edge to edge measurement of the oval of the cross-section). A second diameter (e.g., associated with a minor axis of the stent) may be associated with the stent frame. The second diameter may pertain to a distance across the oval cross-section which bisects the first diameter at a perpendicular angle to the first diameter. The second diameter may be about 20 percent to about 50 percent of the first diameter. However, other sizes and/or shapes are also within the scope of this disclosure.

The stent frame 110 may be configured to be percutaneously delivered to a blood vessel in a compressed configuration. The stent wall 202 may be an open cell wall and/or may be adapted to be secured to a blood vessel wall of a blood vessel through endothelialization and/or using fasteners (e.g., one or more hooks, barbs, and/or other attachment features/means adapted to facilitate secure attachment of the stent frame 110 to the tissue of the target blood vessel wall). When the device 100 is implanted within the blood vessel, the stent frame 110 and/or stent wall 202 of the stent frame 110 may be configured to radially expand into substantially direct surface contact with the blood vessel wall (e.g., the wall of a renal vein). In some embodiments, the stent frame 110 may be configured to be expanded such that the perimeter of a lumen of the stent frame 110 may approximate and/or exceed a perimeter of the blood vessel. In some cases, the stent frame 110 may be expanded to an at least slightly greater perimeter than the native blood vessel to provide improved wall apposition and/or resistance to migration within the blood vessel. Moreover, the stent frame 110 may have a perimeter approximate to and/or greater than the native blood vessel. The perimeter size may be increased to ensure substantial apposition with the blood vessel and/or to maximize a compliance effect. The stent wall 202 and/or a portion of the stent wall 202 may be configured to be endothelialized into the blood vessel wall. In some embodiments, the blood vessel may be a renal vein, and/or the cross-sectional area of the lumen may approximate a cross-sectional area of the renal vein section in which the stent is deployed.

In some examples, the stent frame 110 may be at least partially composed of a shape memory alloy, such as Nitinol®. In some examples, the stent frame 110 may be at least partially composed of cobalt-chrome. In some examples, the stent frame 110 may be at least partially composed of a polymer. In some examples, the stent frame 110 may be at least partially composed of a biodegradable material. In some examples, the stent frame 110 may be at least partially composed of any combination of Nitinol®, cobalt-chrome, a polymer, and/or a biodegradable material.

The stent wall 202 and/or a lumen (defined from the first opening 204 to the second opening (e.g., aperture 118) that is at least partially surrounded by the stent wall 202 may be configured to form a cross-sectional shape defining a cross-sectional area. The shape of the cross-sectional area formed by the stent frame 110 may be elastically deformable between a first configuration and a second configuration. The first configuration may be a substantially circular-shaped cross-sectional shape (e.g., shown by cross-sectional area 220 in FIG. 2D) while the second configuration may be an oval-shaped cross-sectional shape (e.g., shown by cross-sectional area 210 in FIG. 2B or cross-sectional area 213 in FIG. 2C).

In operation, the stent frame 110 may be implanted in a renal vein 102b and may be configured to change the shape of the renal vein 102b when switched between the first configuration and the second configuration. In some examples, the stent frame 110 may be biased toward either the first configuration or the second configuration. For example, the stent frame 110 may be configured to transition the shape/area of a blood vessel from circular/more-circular to non-circular/less-circular shapes, and vice versa, to enhance compliance with respect to an area or vessel portion associated with the implant reshaping. Such stent implant devices/processes may affect vessel reshaping through dynamic reshaping of the structural shape of the stent frame 110. This may produce a change in shape of the blood vessel in which the stent is implanted to produce a change in blood vessel area/volume between the systolic and diastolic phases of the cardiac cycle. For example, for relatively stiff blood vessels, radial outward expansion/stretching of the blood vessel sufficient to achieve a change in volume that produces desirable compliance may not occur as pressure conditions change.

In some examples, the implantation of device 100 may invoke a change in volume of the target blood vessel by changing the structural shape of the stent frame 110. For example, the stent frame 110 may be attached to or endothelialized into the blood vessel wall to pull the wall toward the central axis (C) to modify a portion of the blood vessel from a substantially circular cross-sectional shape (e.g., first configuration) to a substantially oval cross-sectional shape (e.g., second configuration). In some examples, such a change may also change the shape of the aperture 118 of the valve 112 from a substantially circular cross-sectional shape to a substantially oval cross-sectional shape.

FIG. 2B illustrates an example top down view of an example non-circular stent frame 110 for use with the flow modulating devices described herein. The stent wall 202 forms an opening 204 (to the lumen of stent frame 110). The opening 204 may have a cross-sectional area 210 with a major axis 212 that is substantially larger than a minor axis 214. The major axis may run through an axial center of the stent frame 110. For example, the minor axis 214 may be about 20 percent to about 30 percent of the length of the major axis 212. Thus, the cross-sectional shape of opening 204 may be substantially oval (e.g., substantially elliptical).

FIG. 2C illustrates another example top down view of an example non-circular stent frame 110 for use with the flow modulating devices described herein. The stent wall 202 forms the opening 204 (to the lumen of stent frame 110). The opening 204 may have a cross-sectional area 213 with a major axis 216 that is slightly larger than a minor axis 218. The major axis may run through an axial center of the stent frame 110. For example, the minor axis 218 may be about 60 percent to about 80 percent of the length of the major axis 216.

FIG. 2D illustrates a top down view of an example circular stent frame 110 for use with the flow modulating devices described herein. The stent wall 202 forms the opening 204 (to the lumen of stent frame 110). The opening 204 may have a cross-sectional area 220 with a major axis 222 that is substantially the same size as a minor axis 224. For example, the minor axis 224 may be about 75 percent to about 100 percent of the length of the major axis 222.

Thus, the cross-sectional shape of opening 204 may be substantially circular. In such an arrangement, the opening 204 may or may not change shape from the first configuration to the second configuration. For example, the cross-sectional area 220 may remain the same regardless of the configuration. Further for example, the shape of the opening 204 may remain a substantially rigid circle during operating of the device 100. Alternatively, the stent frame 110 may be biased toward an oval and/or other non-circular diastolic configuration. The stent frame 110, when subjected to radially expansive forces, may be configured to transform to a circular systolic configuration shown in FIG. 2D where the minor axis 224 approaches and may equal the major axis 222.

FIG. 2E illustrates an axial view of an example non-circular stent frame 250 for use with the flow modulating devices described herein. The stent frame 250 may be deployable within a blood vessel. The description of the stent frame 250 may be understood to relate to, and/or describe aspects of, any of the stent frames described herein; that is, description of aspects of any example stent frame of the present disclosure may be understood to be implementable in any other example stent frame of the present disclosure.

The stent frame 250 may be formed by a tubular frame wall 252, which may form a wall around a channel 253, thereby defining the channel 253. The stent frame 250 may be an elongate/elongated stent, in that a length (l) (e.g., as shown in FIG. 2A) of the stent is greater than a maximum diameter (d₁) of the stent. A frame wall 252 of the stent frame 250 may be a single, circumferentially-wrapped wall, or may be considered to comprise multiple walls, or wall segments. For example, with respect to oval stents and other non-circular stents, such stents may be considered to comprise sidewall segments 254 that run along relatively long sides of the stent frame 250. Further, sidewall segments 254 may be aligned generally along the length (l) of the stent frame 250, as well as end wall segments 256, which may connect the sidewall segments 254 at one or both ends 258a, 258b of the stent frame 250. The end walls 256 may be outwardly-curved/concave with respect to an axis A of the stent frame 250. The sidewalls 625 may be generally straight over at least a portion of a length thereof, and/or may bow/deflect inward and/or outward, either in a resting, unpressurized state, or in conditions of hoop/wall stress on the frame 631. For example, the sidewalls 625 may bow outward such that the sidewalls 254 are concave from the perspective of the axis (A) of the stent frame 250 and convex from the perspective of the exterior of the stent frame 250.

In the oval configuration, the stent frame 250 may have a cross-sectional area having a major/long axis diameter (d₁) that is substantially larger than the minor/short axis diameter (d₂). For example, the major-axis diameter/dimension (d₁) may advantageously be at least twice as long as the minor-axis diameter/dimension (d₂), or even 3, 4, 5, 6, or 7 times greater. The stent frame 250 may be configured to increase compliance of a blood vessel through constant or near-constant pressure at one or more points along a perimeter/circumference of the blood vessel to cause a change in the perimeter geometry of the vessel. For example, the blood vessel may be changed and/or moved from a substantially non-circular shape to a substantially circular shape.

The dimensions and/or shape of the stent frame 250 may vary based on the particular application and/or target implantation anatomy. For example, the stent length (l) (e.g., shown in FIG. 2A) may be selected to extend over all or a portion of an identified non-compliant length of a target blood vessel. The stent major axis (d₁) and minor axis (d₂), when averaged, may be approximately equal to the diameter of the native blood vessel. For example, for a stent frame configured for deployment in a renal vein, the length (l) may be between about 30 millimeters to about 70 millimeters. In the biased oval/diastolic configuration, the major axis (d₁) may be between about 10 millimeters to about 15 millimeters (or larger/smaller depending on the particular anatomy), and the minor axis (d₂) can be between 20-50 percent of the major axis (d₁). However, other sizes and/or shapes are also within the scope of this disclosure.

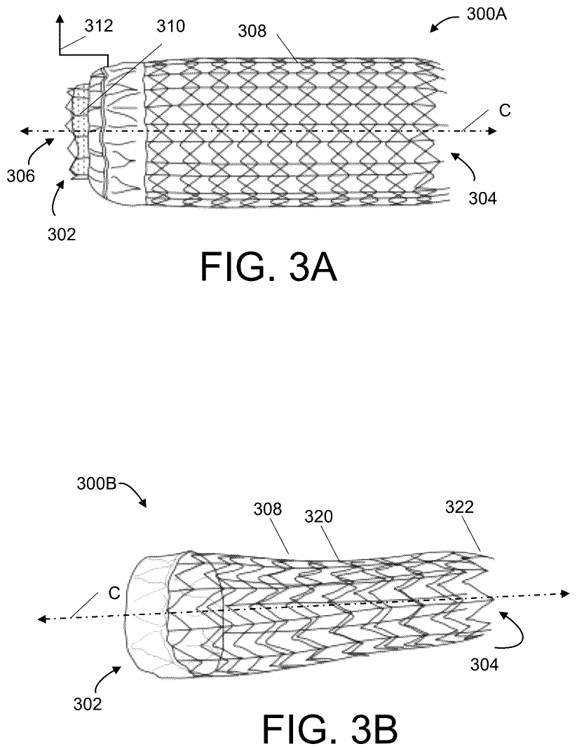

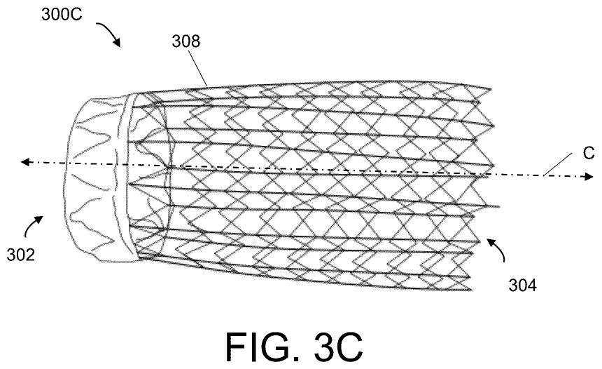

FIGS. 3A-3C illustrate example flow modulating devices in various stages of operation. FIG. 3A depicts a flow modulating device 300A that is substantially elliptical in shape with a substantially elliptical cross section about the central axis (C). In some examples, the device 300A may be substantially flexible such that the shape may take on an irregular perimeter that may conform to a shape of the blood vessel in which the device 300A is installed, for example, when blood flow is provided from an inflow end 302 through to an outflow end 304 through a lumen 306 of the device 300A.

In some examples, the device 300A and stent frame 308 may be adjustable to any number of positions between fully expanded and fully contracted. For example, a portion of the stent frame 308 may collapse inward toward the central axis (C) (e.g., shown in FIG. 3B) and at any intermediate configuration between fully expanded (e.g., shown in FIG. 3C) and fully contracted. In some examples, the stent frame 308 may be expanded or contracted from a particular device state into an expanded position, a partially expanded position, or a partially contracted position.

The inflow end 302 of stent frame 308 may be coupled to a valve 310. The valve 310 may include one or more leaflets (not shown) or other elements that may cover a portion of an aperture associated with the valve to adjust the valve 310 to any number of positions between fully expanded and fully contracted, as described in detail herein. The description of the valve 112 may be understood to relate to, and/or describe aspects of, any of the valves described herein; that is, description of aspects of any example valve of the present disclosure may be understood to be implementable in any other example valve of the present disclosure.

As shown in FIG. 3A, a device 300A is shown in a partially expanded position to allow for engagement and/or endothelization with a blood vessel wall. In this example, the stent frame 308 may allow for a flow of blood through the lumen 306 without substantially hindering a flow of blood through the blood vessel. The expanding or contracting of stent frame 308 may be performed passively as blood flows through the lumen 306 of device 300A. In some examples, the expanding or contracting of stent frame 308 may be performed actively in response to a control input received from an optional control element 312. For example, a portion of the stent frame 308 or valve 310 may include or be coupled to the control element 312 to cause the stent frame 308 to adjust to any number of positions between fully expanded and fully contracted. In some examples, the control element 312 may instead be configured to cause the valve 310 to adjust to any number of positions between fully expanded and fully contracted.

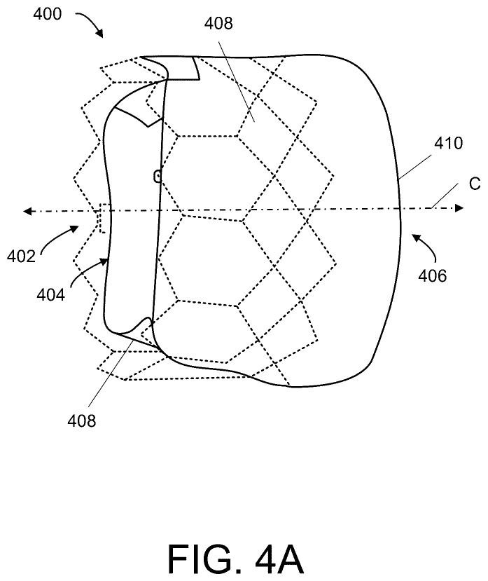

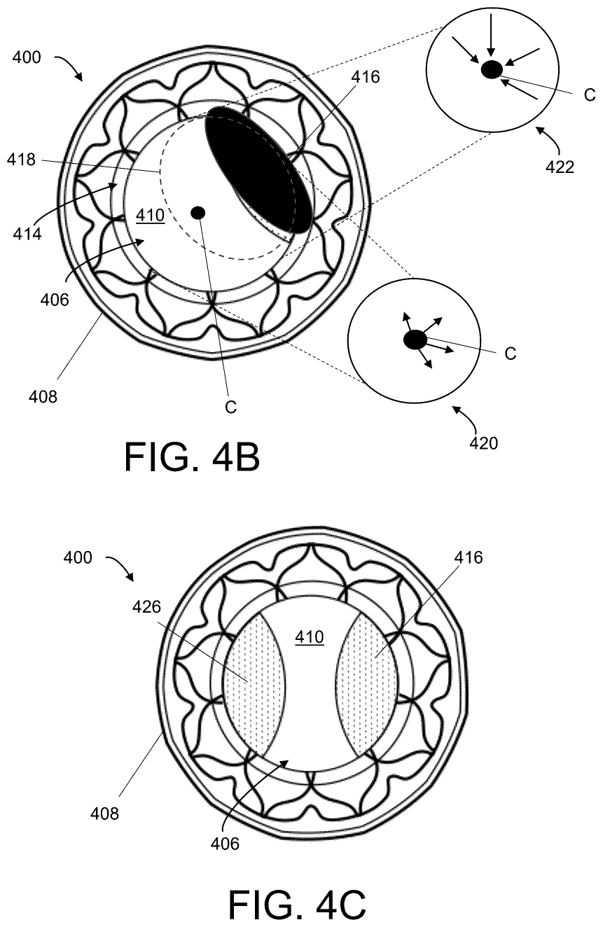

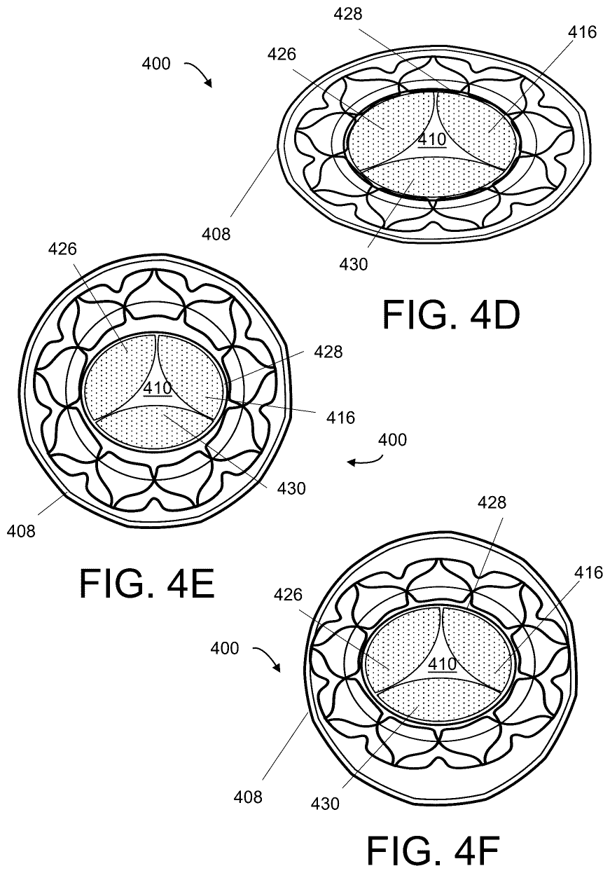

For example, the control element 312 may be configured to move at least one leaflet (e.g., as shown in FIGS. 4B-4E) between a first position and a second position to manipulate the valve 400 between an unrestricted blood flow position and a restricted blood flow position. The control element 312 may be actuated by an actuation device (not shown) to radially expand (e.g., unrestricted blood flow position) and constrict (uniformly or nonuniformly) (e.g., restricted blood flow position) a portion of the aperture associated with lumen 306 at inflow end 302, which may allow the valve 310 to function as an adjustable blood flow restrictor.

FIG. 3B illustrates the device 300B in a partially contracted position to narrow the stent frame 308 and a respective portion of a blood vessel at a location (e.g., from location 320 to location 322) along the stent frame 308, which may move the stent frame 308 and blood vessel toward a portion of the central axis (C). Other locations and portions of stent frame 308 may instead be narrowed, and locations 320 and 322 are merely examples. In general, the partially contracted position may allow partial occlusion of the blood vessel. For example, when the device 300B is implanted in a blood vessel and is configured in the partially contracted position, the device 300B may allow a partial amount of blood to flow from the inflow end 302 through to the outflow end 304 and may hinder a flow of the blood flow by a predefined amount associated with a cross-sectional area formed when a portion of the stent frame 308 is partially contracted (e.g., partially collapsed, partially expanded).

FIG. 3C depicts a device 300C in an expanded position. The expanded position depicts the stent frame 308 expanded which may allow blood to flow from the inflow end 302 through to the outflow end 304 without substantially hindering the blood flow speed or the blood flow amount.

FIG. 4A illustrates a side perspective view of an example valve 400 for use with the flow modulating devices described herein. The valve 400 may function to modulate blood flow through a blood vessel, such as a renal vein. As shown in FIG. 4A, the valve 400 may define or include an inflow end 402 with a support portion 404, and an outflow end 406. The inflow end 402 may be coupled to the stent frame 408 (e.g., similar to stent frames 110, 250, 308, etc.). The outflow end 406 may define an aperture 410. The valve 400 may also include one or more leaflets as described in detail herein that may be positionable to overlay at least a portion of the aperture 410. The inflow end 402 and, in particular, the support portion 404 may be configured to couple to one or more leaflets (not shown). In some examples, the valve 400 may comprise or be formed of one or more of: a polymer, a biomaterial, or a textile.

The description of the valve 400 may be understood to relate to, and/or describe aspects of, any of the valves described herein; that is, description of aspects of any example valve of the present disclosure may be understood to be implementable in any other example valve of the present disclosure.

FIG. 4B illustrates a top down view of the example valve 400 for use with the flow modulating devices described herein. The valve 400 may function to modulate blood flow through a blood vessel, such as a renal vein. As shown, the valve 400 may define or include the outflow end 406 with a support portion 414. A perimeter of the outflow end 406 may be coupled to the stent frame 408. The aperture 410 may be defined in a center portion through the valve 400. The valve 400 may also include one or more leaflets (e.g., leaflet 416) that may be positionable to overlay at least a portion of the aperture 410. The outflow end 406 and, in particular, the support portion 414 may be configured to couple to one or more leaflets 416. In general, the valve 400 may include at least one leaflet 416 at the outflow end 406.

In some examples, the leaflet 416 may be configured to move in a radial direction that is substantially perpendicular to a central axis (C) (shown in FIG. 4A) of the stent frame 408. For example, the valve 400 may be triggered by passive blood flow or by active (e.g., mechanical or electronic) signals received from a control element (not shown) to cause the leaflet 416 to move in the radial direction to reduce a cross-sectional area of the aperture 410 and restrict flow through the valve 400. For example, the control element may be coupled to the valve 400 such that actuation of the control element causes the leaflet 416 to move between a first position (e.g., as shown by leaflet 416) and a second position (e.g., as shown by leaflet outline 418) to manipulate the valve 400 between an unrestricted blood flow position and a restricted blood flow position, respectively. For example, an active or passive trigger may move the leaflet 416 to partially occlude aperture 410 (or partially un-occlude aperture 410) in response to an existence of an elevated pressure within the blood vessel. In some examples, the active or passive trigger to move the leaflet 416 may be in response to detecting, via a sensor (not shown) associated with valve 400, an elevated pressure within the blood vessel. In some examples, the leaflet 416 may instead be moveably coupled (e.g., hinged) to a location on the support portion 414.

In some examples, the unrestricted blood flow position may include a position in which the at least one leaflet 416 is moved radially away from the central axis (C) of the stent frame 408 to cause an increase in blood flow through the valve 400, as shown by the arrow directions of zoomed-in view 420. In particular, the first position comprises the at least one leaflet 416 at least partially obstructing the aperture 410. In some examples, the restricted blood flow position may include a position in which the at least one leaflet 416 is moved radially toward the central axis (C) of the stent frame 408 to cause a decrease in blood flow through the valve, as shown by the arrow direction of zoomed-in view 422. In particular, the second position comprises the at least one leaflet 416 being arranged so that the aperture 410 is substantially unobstructed.

FIG. 4C illustrates a top down view of an example valve 400 for use with the flow modulating devices described herein. The valve 400 shown in FIG. 4C includes the first leaflet 416, as described above as well as a second leaflet 426. The leaflets 416 and 426 are positionable to overlay at least a portion of the aperture 410 according to a selected mode for the device in which the valve 400 is installed. FIG. 4D illustrates a top down view of an example valve 400 for use with the flow modulating devices described herein. The valve 400 shown in FIG. 4D includes the first leaflet 416, the second leaflet 426, as described above as well as a third leaflet 430. Here, a perimeter 428 (and cross-sectional area) of the valve 400 is oval in shape. In addition, the perimeter (and cross-sectional area) of the stent frame 408 is also oval. The leaflets 416, 426 and 430 are positionable to overlay at least a portion of the aperture 410 according to a selected mode for the device in which the valve 400 is installed. The leaflets 416, 426, and 430 may be elongated to account for the oval shape of the frame 408 and/or the valve 400. FIG. 4E illustrates a top down view of an example valve for use with the flow modulating devices described herein. The valve 400 shown in FIG. 4E includes the first leaflet 416, the second leaflet 426, and the third leaflet 430. Here, the perimeter 428 (and cross-sectional area) of the valve 400 is substantially oval in shape while the perimeter (and cross-sectional area) of the stent frame 408 is substantially circular in shape. The leaflets 416, 426 and 430 are positionable to overlay at least a portion of the aperture 410 according to a selected mode for the device in which the valve 400 is installed. The leaflets 416, 426, and 430 may be elongated to account for the oval shape of the valve 400.

FIG. 4F illustrates a top down view of an example valve for use with the flow modulating devices described herein. The valve 400 shown in FIG. 4F includes the first leaflet 416, the second leaflet 426, and the third leaflet 430. Here, the perimeter 428 (and cross-sectional area) of the valve 400 and the perimeter (and cross-sectional area) of the stent frame 408 are substantially oval in shape. The leaflets 416, 426 and 430 are positionable to overlay at least a portion of the aperture 410 according to a selected mode for the device in which the valve 400 is installed. The leaflets 416, 426, and 430 may be elongated to account for the oval shape of the valve 400.

Although the valve 400 is described with one, two, or three leaflets, additional leaflets are possible. For example, the valve 400 may have three to five leaflets; four to six leaflets, etc. In operation, the one or more leaflets are configured to proportionally move toward the central axis of the stent frame in response to an elevated pressure within the blood vessel to restrict flow through the valve 400.

In some examples, a flow modulating device (e.g., a stent frame 408 with a valve 400) can include one or more passive mechanisms to modulate flow through the device. In some examples, a passive mechanism can include more than one mode of operation. For example, a valve 400 (or member or component) of a blood flow regulator (or modulator or restrictor) described herein can move in a particular way in response to a first externally applied force, and then the valve 400 (or member or component) of a blood flow regulator (or modulator or restrictor) described herein can move in a different way in response to a second externally applied force. In some cases, the first externally applied force and the second externally applied force are applied from the same external force (e.g., blood pressure), with different quantitative ranges. For example, a leaflet, flap, or valve (e.g., used to restrict blood flow through a blood vessel) can move in a first mode in response to an increase in blood pressure within a first blood pressure range, and move in a second mode in response to an increase in blood pressure within a second blood pressure range that is different (e.g., higher) than the first blood pressure range. The first mode can be that an end of the leaflets, flaps, or valve portions move towards one another (or toward a center of the aperture 410) to further restrict blood flow within the blood vessel, and the second mode can be that the leaflets, flaps, or valve portions prolapse, thereby moving away from one another (or away from the center of the aperture 410) to increase the blood flow within the blood vessel.

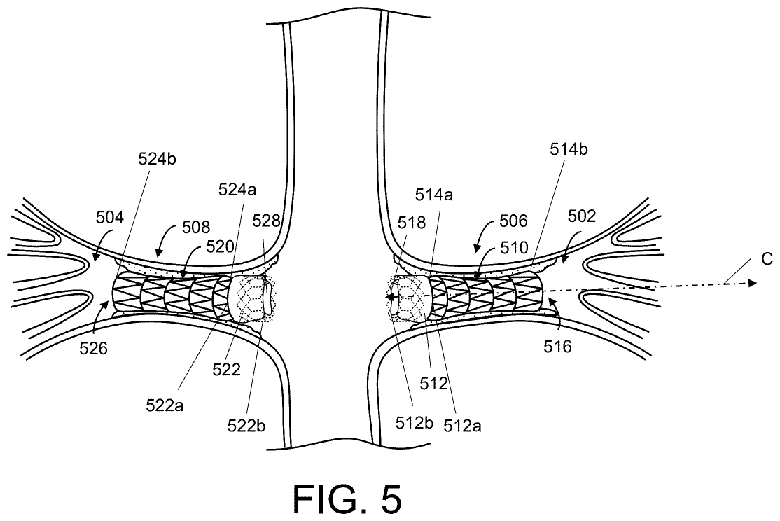

FIG. 5 illustrates a side view of two example flow modulating devices implanted in a left renal vein 502 and a right renal vein 504, respectively. For example, a flow modulating device 506 is shown implanted in the left renal vein 502 and a flow modulating device 508 is shown implanted in the right renal vein 504. The devices 506, 508 may represent prosthetic implants that are configured to modulate blood flow through the blood vessel in which the devices 506, 508 are respectively implanted. For example, the devices 506, 508 may improve outflow from the kidneys to improve function of the kidneys. For example, outflow may be improved when the devices 506, 508 function to modulate a volume of blood flowing from the respective kidney to the inferior vena cava to reduce and/or otherwise modulate venous pressure. The devices 506, 508 may be implanted in respective renal veins 502, 504 to improve flow from one or both kidneys to the IVC. In some examples, the devices 506, 508 may represent transcatheter-deliverable devices configured to operate in a manner that minimizes risk of thrombosis.

As shown in FIG. 5, the device 506 includes a stent frame 510 and a valve 512. The stent frame 510 may be coupled to the valve 512. The stent frame 510 may be a self-expanding or balloon expandable frame that may be delivered into the blood vessel (e.g., via femoral access, jugular access, subclavian access, or transfemoral access) using a sheathed catheter (not shown).

In general, the stent frame 510 may be an elongated and substantially tubular member including a first end 514a with a first opening (covered by valve 512). The stent frame 510 may further include a second end 514b with a second opening 516, a lumen extending between the first opening and the second opening, and/or a stent length extending between the first end 514a and the second end 514b. In some examples, the stent frame 510 may have a non-circular cross-sectional shape. For example, the stent frame 510 (and lumen) may be substantially tubular-shaped with a substantially oval (e.g., elliptical) cross-sectional shape about a central axis (C). In some examples, the stent frame 510 may have a substantially circular cross-sectional shape. For example, the stent frame 510 may be substantially tubular-shaped with a substantially circular cross-sectional shape about the central axis (C). The cross-sectional shape of the stent frame 510 may further be any shape, including a triangle, a peanut, a figure-8, and/or a kidney shape. In general, the shapes of the stent frame described herein may refer to a shape of an axial cross-section of the stent frame, as depicted in the view of FIG. 2E.

The stent frame 510 may be configured to receive the valve 512. For example, the stent frame 510 may be coupled to the valve 512. In some examples, the stent frame 510 may be removably coupled to the valve 512. In some examples, the stent frame 510 may be coupled to the valve by one or more elastomeric members representing at least one tubular structure coupled to both the stent frame 510 and the valve 512, as discussed in detail throughout this disclosure.

The valve 512 may define an inflow end 512a and an outflow end 512b. The inflow end 512a may be coupled to the stent frame 510 at first end 514a. The outflow end 512b may define the aperture 518 that may be configured to open or partially open (e.g., or partially close) to modulate blood flow therethrough. For example, the device 506 may be triggered to reduce a cross-sectional area of the aperture 518 to restrict flow through the valve 512 in response to elevated pressure within the blood vessel. The valve 512 may have a diameter of about 15 millimeters to about 25 millimeters.

Referring again to FIG. 5, the device 508 includes a stent frame 520 and a valve 522. The stent frame 520 may be coupled to the valve 522. The stent frame 520 may be a self-expanding or balloon expandable frame that may be delivered into the blood vessel (e.g., via femoral access, jugular access, subclavian access, or transfemoral access) using a sheathed catheter (not shown).

In general, the stent frame 520 may be an elongated and substantially tubular member including a first end 524a with a first opening (covered by valve 522). The stent frame 520 may further include a second end 524b with a second opening 526, a lumen extending between the first opening and the second opening, and/or a stent length extending between the first end 524a and the second end 524b. In some examples, the stent frame 520 may have a non-circular cross-sectional shape. For example, the stent frame 520 (and lumen) may be substantially tubular-shaped with a substantially oval (e.g., elliptical) cross-sectional shape. In some examples, the stent frame 520 may have a substantially circular cross-sectional shape. For example, the stent frame 520 may be substantially tubular-shaped with a substantially circular cross-sectional shape. The cross-sectional shape of the stent frame 520 may further be any shape, including a triangle, a peanut, a figure-8, and/or a kidney shape.

The stent frame 520 may be configured to receive the valve 522. For example, the stent frame 520 may be coupled to the valve 522. In some examples, the stent frame 520 may be removably coupled to the valve 522. In some examples, the stent frame 520 may be coupled to the valve by one or more elastomeric members representing at least one tubular structure coupled to both the stent frame 520 and the valve 522, as discussed in detail throughout this disclosure.

The valve 522 may define an inflow end 522a and an outflow end 522b. The inflow end 522a may be coupled to the stent frame 520 at first end 524a. The outflow end 522b may define the aperture 528 that may be configured to open or partially open (e.g., or partially close) to modulate blood flow therethrough. For example, the device 508 may be triggered to reduce a cross-sectional area of the aperture 528 to restrict flow through the valve 522 in response to elevated pressure within the blood vessel.

In operation, the valves 512, 522 can be operated to improve a pressure gradient across the kidneys by protecting blood outflow from the kidneys from high central venous pressures. For example, the valves 512, 522 may be one-way valves configured to modulate blood flow from at least one organ (e.g., a kidney). The valves 512, 522 may be triggered to close in response to pressure detected within the IVC (or a related vein) being greater than a predefined threshold pressure. In some examples, the valves 512, 522 may be configured to restrict blood flow through an aperture 518 or aperture 528 in response to an elevated pressure within the blood vessel in which the device 506 and/or device 508 is implanted.

The description of the stent frame 510 and stent frame 520 may be understood to relate to, and/or describe aspects of, any of the stent frames described herein; that is, description of aspects of any example stent frame of the present disclosure may be understood to be implementable in any other example stent frame of the present disclosure.

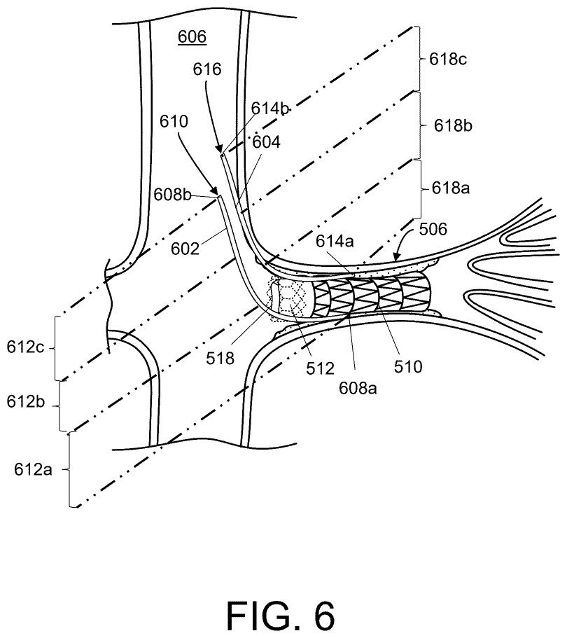

FIG. 6 illustrates a side view of an example flow modulating device 506 implanted in a right renal vein 502. The device 506 includes the stent frame 510 and the valve 512 with an aperture 518, similar to the embodiment shown in FIG. 5. The device 506 in this example includes a first elastomeric member 602 and a second elastomeric member 604. Each elastomeric member 602, 604 may be a substantially elongate extension or tubular extension leading from an inflow end (e.g., opening 608a) of the device 506 disposed within the renal vein 502 to a narrower outflow end (e.g., opening 608b) of the device 506 disposed near to or within the IVC 606.

In operation, the elastomeric members 602, 604 may provide for a Venturi effect within the members 602, 604. For example, the members 602, 604 may narrow in diameter between the inflow end (e.g., opening 608a) and the outflow end (e.g., opening 608b), thereby accelerating blood flow velocity therethrough as the cross-sectional area converges. Such a design may lead to a lower pressure at the outflow end due to the increased velocity of the blood through the member 602.

The first elastomeric member 604 has a first opening 608a and a second opening 608b and a first lumen 610 is defined therebetween. The first opening 608a is coupled to a portion of the stent frame 510. The elastomeric member 602 may be configured to accelerate a flow velocity of blood through the first lumen 610 and lower a flow pressure associated with the blood vessel.

The first lumen 610 may define a first cross-sectional area at the first opening 608a that extends for a first portion 612a of a length of the member 602, a second cross-sectional area that extends over a second portion 612b of the length of the member 602, and a third cross-sectional area that extends over a third portion 612c of the length of the member 602. The first cross-sectional area of portion 612a is greater than the second cross-sectional area of portion 612b. The second cross-sectional area of portion 612b is greater than the third cross-sectional area of portion 612c.

While portions 612a, 612b, and 612c are shown having a specific length of member 602, other lengths and divisions of member 602 are possible. For example, a length of the portion 612a may match a length of portion 612c while a length of portion 612b is longer than portions 612a, 612c. In some examples, each portion 612a, 612b, and 612c each have a different length. In some examples, a length of portion 612c is longer than a length of both of portion 612a and 612b combined.

The second elastomeric member 604 has a third opening 614a and a fourth opening 614b and a second lumen 616 is defined therebetween. The third opening 614a is coupled to a portion of the stent frame 510. The elastomeric member 604 may be configured to accelerate a flow velocity of blood through the second lumen 616 and lower a flow pressure associated with the blood vessel.

The second lumen 616 may define a fourth cross-sectional area at the third opening 614a that extends for a first portion 618a of a length of the member 604, a fifth cross-sectional area that extends over a second portion 618b of the length of the member 604, and a sixth cross-sectional area that extends over a third portion 618c of the length of the member 604. The fourth cross-sectional area of portion 618a is greater than the fifth cross-sectional area of portion 618b. The fifth cross-sectional area of portion 618b is greater than the sixth cross-sectional area of portion 618c.

While portions 618a, 618b, and 618c are shown having a specific length of member 604, other lengths and divisions of member 604 are possible. For example, a length of the portion 618a may match a length of portion 618c while a length of portion 618b is longer than portions 618a, 618c. In some examples, each portion 618a, 618b, and 618c each have a different length. In some examples, a length of portion 618c is longer than a length of both of portion 618a and 618b combined.

In some examples, a length of elastomeric member 602 matches a length of elastomeric member 604. In some examples, a length of elastomeric member 602 is shorter than a length of elastomeric member 604. In some examples, a length of elastomeric member 602 is longer than a length of elastomeric member 604. In some examples, a length of elastomeric member 602 is about 50 percent to about 75 percent of a length of elastomeric member 604. In some examples, a length of elastomeric member 602 is about 25 percent to 50 percent of a length of elastomeric member 604.

Example cross-sectional areas for the elastomeric members 602, 604 may include about 8 millimeters to about 15 millimeters. For example, the cross-sectional area of the elastomeric members 602, 604 may be tapered (e.g., an overall reduction in cross-sectional area from a first end 608a, 614a to a second end 608b, 614b) to result in cross-sectional areas from about 15 millimeters to about 10 millimeters; from about 15 millimeters to about 8 millimeters; from about 12 millimeters to about 10 millimeters; from about 12 millimeters to about 8 millimeters. In some examples, the cross-sectional areas of elastomeric members 602, 604 are not tapered, but instead have a uniform cross-sectional area from the first end to the second end.

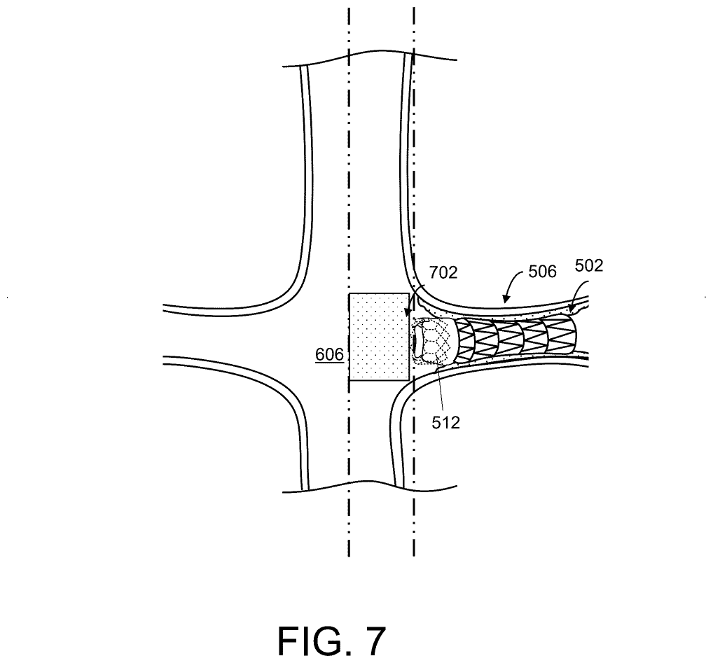

FIG. 7 illustrates a side view of an example flow modulating device 700 implanted in a right renal vein. In this example, a single stent device (e.g., device 506) is implanted based on a determined decline in function of the right kidney and/or a detected blood pressure outside of a predetermined range for a blood pressure associated with the IVC and/or one or more veins leading to the right kidney. The device 506 may include at least one valve 512, as described in detail throughout this disclosure. While device 506 is depicted within the renal vein 502, the device may instead be placed further into the IVC 606 within gap 702 and with a portion of the device 506 being implanted within the renal vein 502 while a portion of the device 506 is partially within the IVC 606.

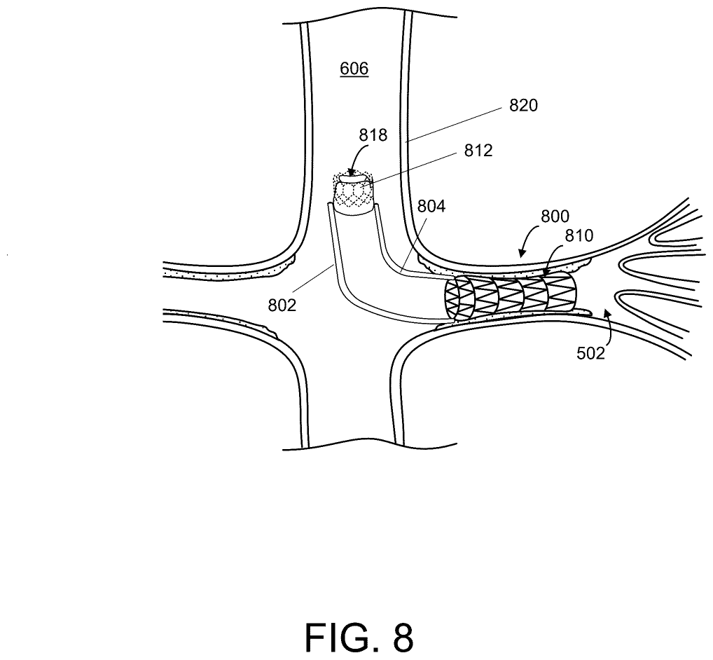

FIG. 8 illustrates a side view of an example flow modulating device 800 implanted in a right renal vein. The device 800 includes the stent frame 810 and a valve 812 with an aperture 818 and a first elastomeric member 802 and a second elastomeric member 804, similar to the device 506 of FIG. 6. In this example, the valve 812 is coupled to the stent frame 810 via elastomeric member 802 and elastomeric member 804. The valve 812 is shown within a center portion of the IVC 606, however, the valve may instead be attached anywhere along vessel wall 820. For example, the valve 812 may be held in place by an additional frame (e.g., a stent frame or partial stent frame) allowing blood flow through the frame and to the heart while preventing misalignments or unwanted blocking of the valve 812 that could be caused by an extreme condition (e.g., blood pressure drop, blood pressure rise, heart arrythmias, etc.). Similarly, although members 802, 804 are depicted floating within the IVC 606, one or both members 802, 804 may be anchored along vessel wall 820 and/or along stent frame 810.

The valve 812 may have a diameter that is larger than that of the diameter of valve 512, for example, because the valve 812 may be placed in the larger IVC 606 rather than placed in the smaller renal vein 502. For example, the valve 812 may have a diameter of about 20 millimeters to about 40 millimeters. Accordingly, the valve 812 may be configured to attach to similar elastomeric members 602, 604 or may accommodate slightly larger elastomeric members given the increase in valve size.

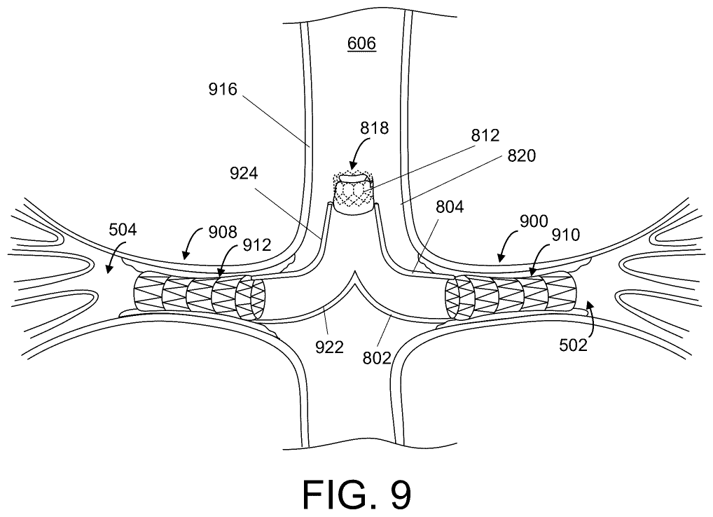

FIG. 9 illustrates a side view of example flow modulating devices implanted in a left renal vein 502 and a right renal vein 504 and a portion of the inferior vena cava 606. For example, a first device 900 is implanted in the right renal vein 502 and a second device 908 is implanted in the left renal vein 504.

The device 900 includes the stent frame 810 and a valve 812 with an aperture 818, and a first elastomeric member 802 and a second elastomeric member 804. In this example, the valve 812 is coupled to the stent frame 810 via elastomeric member 804. The valve 812 is shown within a center portion of the IVC 606, however, the valve 812 may instead be attached to wall 820 or wall 916. In some examples, the valve 812 remains floating within IVC 606, but may be placed along the IVC 606 based on a length of one or more members 802, 804. Similarly, although members 802, 804 are depicted floating within the IVC 606, one or both members 802, 804 may be anchored along vessel wall 820 and/or along stent frame 810.

The device 920 includes the stent frame 912 and the valve 812 with an aperture 818, and an elastomeric member 922 and an elastomeric member 924. In this example, the valve 812 is coupled to the stent frame 912 via elastomeric member 924. The valve 812 is shown within a center portion of the IVC 606, however, the valve 812 may instead be attached to wall 820 or wall 916. In some examples, the valve 812 remains floating within IVC 606, but may be placed along the IVC based on a length of one or more members 922, 924. Similarly, although members 922, 924 are depicted floating within the IVC 606, one or both members 922, 924 may be anchored along vessel wall 916 and/or along stent frame 912.

The elastomeric members 802, 804, 922, 924 may function together to reduce pressure within the renal veins 502, 504, and/or the IVC 606. The elastomeric member 802 is shown at least partially coupled along an outside edge of elastomeric member 922. Such a coupling can allow a flow of blood from the veins 502, 504 to be guided through the valve 812 to ease pressure within the IVC 606 and/or veins 502, 504. In some examples, the member 802 and member 922 may instead be uncoupled and flowing freely within the IVC 606. In some examples, the member 802 and member 922 may be coupled to the valve 812.

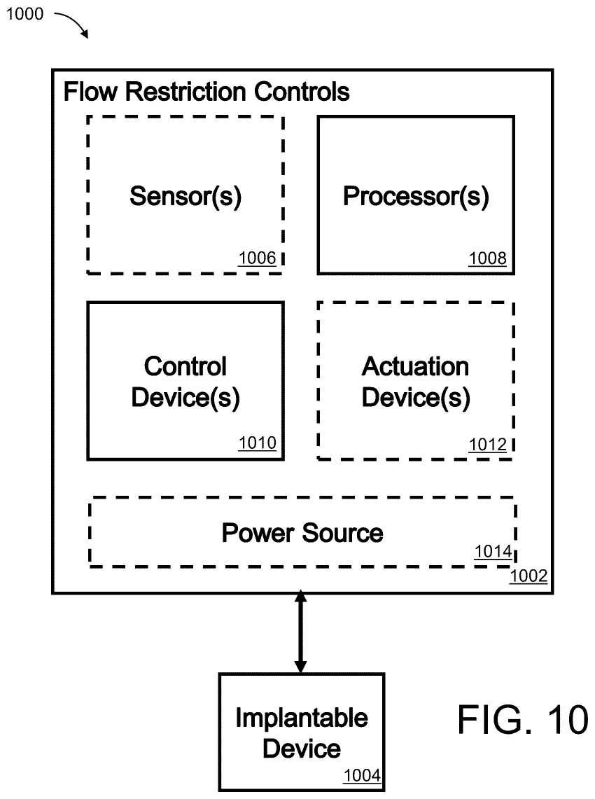

FIG. 10 is a block diagram of an example system 1000 for modulating blood flow through one or more blood vessels. The system 1000 may be used with any of the flow modulating devices described herein (e.g., devices 100, 300A, 300B, 300C, 400, 506, 508, 700, 800, 900, 908, 1300, etc.) As shown, the system 1000 includes flow restriction controls 1002 and at least one implantable device 1004. The implantable device 1004 may correspond to any of the flow modulating devices described herein.

The flow restriction controls 1002 may include one or more optional sensors 1006, one or more processors 1008, one or more control devices 1010, and one or more optional actuation devices 1012. Optionally, the flow restriction controls may include a power source 1014 that may be internal to the controls 1002, internal to the implantable device 1004, or external to both the flow restriction controls 1002 and the implantable device 1004. In some examples, the power source may be wired to flow restriction controls 1002 or implantable device 1004. In some examples, the power source may be remotely accessed (e.g., wirelessly) by flow restriction controls 1002 or implantable device 1004.

The optional sensors 1006 may generally function to sense (e.g., detect) properties of the blood in which the sensor(s) are disposed within. For example, the optional sensors 1006 may detect blood pressure within the blood vessel and/or any other physiological or anatomical parameters or properties of the blood or blood vessel. The optional sensors 1006 may include one or more of an image sensor, a strain gauge, a piezoelectric sensor, a capacitance sensor, and/or a vacuum pressure sensor. In general, sensor signals from sensors 1006 may be transmitted to control devices 1010 or elements described herein via a wired or wireless connection. Additionally, and optionally, the sensors 1006 may utilize one or more processors 1008 to transmit data to remote computing devices. The transmitted data may include sensor measurements, device position data and/or statistics, actuation events, or any other data from the system.

The processors 1008 may include one or more microprocessors, microcontrollers, or the like, as described elsewhere herein. The control devices 1010 may include active or passive controls including, but not limited to wires, sutures, operated switches, motor controllers, and/or antennas. In some examples, the control devices 1010 may include external control devices including, but not limited to remote computers, tablets, smart phones, and/or external control devices for powering and/or controlling the flow restriction controls 1002.

The optional actuation devices 1012 may include mechanically actuation devices, electrically actuated devices, electromechanically actuated devices, or a combination thereof. For example, actuation devices 1012 may include any one or more of a wire, a suture, a pull wire, a linear actuator (e.g., a pneumatic linear actuator, an electromechanical linear actuator, or a hydraulic linear actuator), a magnet or coil, etc. The power sources 1014 may include, but are not limited to, battery power, wall power, magnets, induction coils, or the like.

In operation of system 1000, the actuation device 1012 may be coupled to the control device 1010, which may manipulate or move portions (e.g., valves, leaflets, stent frames, etc.) of the implantable device 1004 based on one or more signals received from a sensor 1006. In embodiments that utilize a processor 1008, the processor 1008 may be communicatively coupled to sensors 1006, control devices 1010, actuation devices 1012, power source 1014, and/or implantable device 1004 to actuate the implantable device 1004 into a restricted blood flow state, an unrestricted blood flow state, or any position therebetween.

The system 1000 may be included within or be coupled to any of the devices described herein (e.g., devices 100, 300A, 300B, 300C, 400, 506, 508, 700, 800, 900, 908, 1300, etc.). For example, such devices may include at least one sensor 1006 coupled to the implantable device (e.g., devices 100, 300A, 300B, 300C, 400, 506, 508, 700, 800, 900, 908, 1300, etc.) where the sensor 1006 is configured to detect a blood pressure. Such devices may also include or be coupled to a processor 1008 coupled to the at least one sensor 1006 and a control element (e.g., control device 1010 and/or actuation device). The control element may be coupled to any or each of the valves of the device, the at least one sensor 1006, and the processor 1008.

In some examples, the control element (e.g., control device 1010 and/or actuation device 1012) is configured to move one or more leaflets of the valve to an unrestricted blood flow position when the sensor 1006 detects a blood vessel pressure below a predefined pressure level. Further, the control element may also be configured to move one or more leaflets of the valve to a restricted blood flow position when the sensor 1006 detects the blood vessel pressure is at or above the predefined pressure level, as described in detail herein.

### Methods

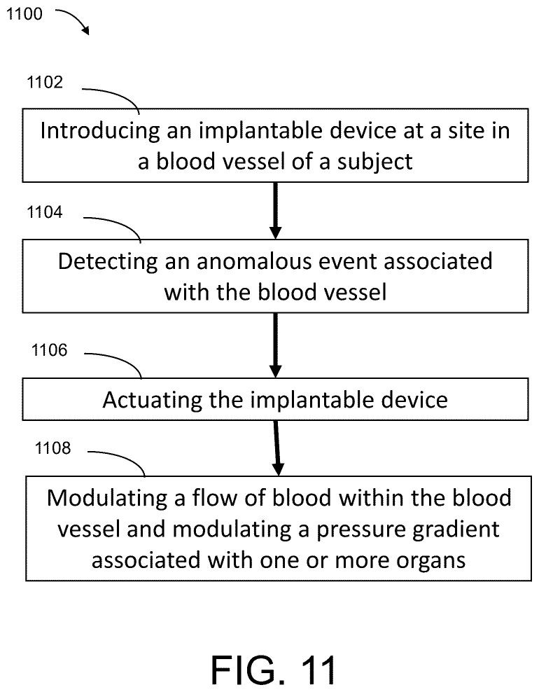

FIG. 11 is a flow diagram of an example process 1100 for improving a pressure gradient across the kidneys. In particular, the process 1100 may modulate a pressure gradient and/or a flow of blood within a blood vessel of a first kidney and/or a second kidney of a subject. The process 1100 functions to reduce pressure in one or more renal veins and/or pressure within the IVC. In general, process 1100 may be used with any of the devices described in FIGS. 1-10 above. In some examples, the process 1100 may be a method of treatment for modulating blood flow and/or a pressure gradient across the kidneys in a subject having chronic kidney disease.

As an example, the device used with process 1100 may include a stent frame with an oval-shaped cross-sectional area and a valve defining an inflow end and an outflow end. The valve may comprise or be formed of one or more of: a polymer, a biomaterial, or a textile. In some examples, the valve may include one or more additional leaflets arranged with the at least one leaflet. The at least one leaflet and the one or more additional leaflets may be configured to move toward the central axis of the stent frame to restrict flow through the valve when there is an elevated pressure within the renal vein, as shown in at least FIG. 1.

The inflow end may be coupled to the stent frame and the outflow end may define an aperture having a cross-sectional area with a diameter of about 5 millimeters to about 20 millimeters. The valve may include at least one leaflet at the outflow end of the valve where the at least one leaflet is positionable to overlay at least a portion of the aperture and configured to move in a radial direction that is perpendicular to a central axis of the stent frame. Optionally, the device may include one or more elastomeric members coupled to the stent frame, as described in detail herein.

The process 1100 may include introducing a vessel occlusion device (e.g., device 100, 300A-300C, 400, 506, 508, 700, 800, 900, 908, or 1300) at a site in a blood vessel (e.g., a renal vein) of a subject (block 1102). For example, the devices described herein may be partially or fully housed by a stent frame (e.g., a stent). The frame housing the device 100, for example, may be introduced to a blood vessel or tissue site using a delivery system. In a renal procedure, a catheter tip and/or catheter may be configured to pass from the IVC into a renal vein to implant the device 100. Further, for central venous access, a catheter tip and/or catheter may be configured to pass from the femoral vein into the IVC to implant the device into a portion of the renal vein and/or a portion of the IVC.

At block 1104, the process 1100 may include detecting, by the device, an anomalous event (or several events) associated with the blood vessel. For example, the process 1100 may include detecting, by one or more device sensors, an increasing blood pressure in the IVC and/or in a left or right renal vein.

At block 1106, the device may be actuated based on the sensed anomalous event (or several events). Actuating the device may trigger modulation of a flow of blood within the blood vessel site based on the detected anomalous event. For example, actuating the device may cause a partial occlusion of the blood in the blood vessel at the site of the device. For example, if the device is implanted into the renal vein of a subject having chronic kidney disease, the device can be actuated to modulate a volume of blood flowing from the blood vessel into a kidney to decrease pressure within the renal vein, and/or to modulate a pressure gradient associated with the kidney or both kidneys.

For example, actuating the device to modulate a flow of blood within the renal vein and/or the IVC may include causing the at least one leaflet (of the valve associated with the device) to move in the radial direction to increase or decrease a cross-sectional area of the aperture of the device. Increasing the cross-sectional area of the aperture results in unrestricting flow through the valve. Decreasing the cross-sectional area of the aperture results in restricting flow through the valve.

### Example Implantation of Flow Modulating Devices

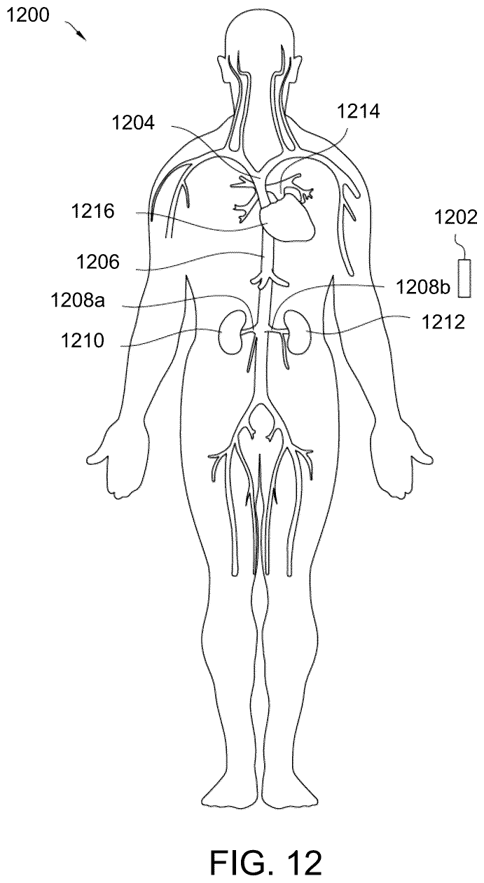

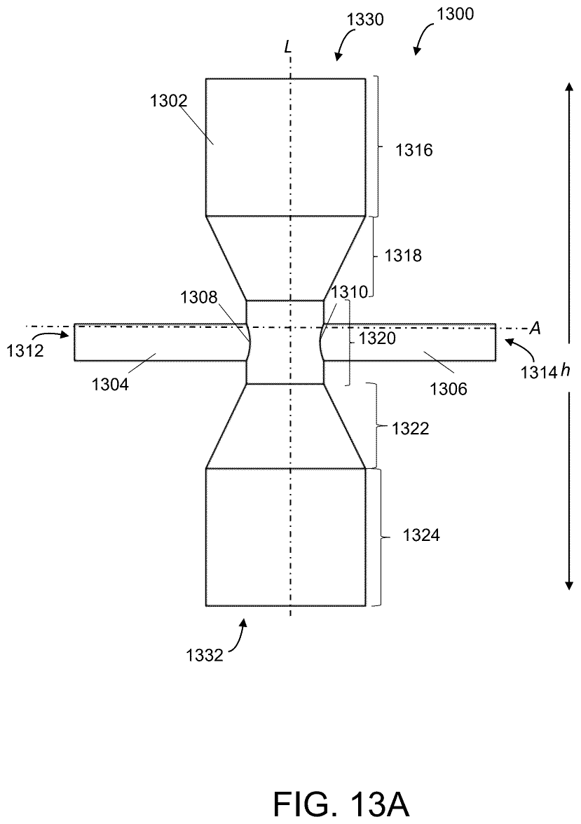

FIG. 12 illustrates a schematic representation of portions of a subject 1200. The flow modulating devices described herein of FIGS. 1-10 (represented in FIG. 12 by device 1202) may be introduced (e.g., implanted) in vasculature of the body. In general, the device 1202 may represent any of the flow modulating devices described herein (e.g., 100, 300A-300C, 400, 506, 508, 700, 800, 900, 908, 1300, etc.) and may include the same or similar functionality and/or structures. In some examples, the device 1202 may be implanted in or near to a portion of the IVC 1206. The subject 1200 is illustrated with a representation of a portion of the vasculature system to generally illustrate the SVC 1204 and the IVC 1206 within the subject 1200. However, it is to be understood that no dimensions or relative sizes of components may be inferred from the relative sizes and dimensions of elements in the figures.

The subject 1200 includes a number of vessels and organs that may circulate blood throughout the body. For example, renal veins 1208a and 1208b drain blood from a respective right kidney 1210 and left kidney 1212. Renal veins 1208a and 1208b connect to the IVC 1206. Blood from the aorta 1214 flows to the IVC 1206. Blood travels from the aorta 1214 of heart 1216 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 1206 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles. Heart failure patients can have multiple comorbidities that cause excessive congestion or accumulation of blood volume in the splanchnic venous circulation. The excessive congestion or accumulation causes excess load on the heart, over-reactive fight or flight responses, poor oral medication absorption, etc. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized. For example, because blood flow from the splanchnic venous circulation is directed through hepatic veins and into the IVC 1206, devices (as described herein) may be placed into the IVC 1206 and/or one or more renal veins 1208a, 1208b to limit blood flow to allow the splanchnic venous circulation to expand with increased blood volume. This may also allow the body to accumulate blood volume in the splanchnic venous circulation, which can maximize the downstream drop of pressure relative to upstream increase of pressure.

In some examples, the device 1202 may enable modulating and/or balancing of blood flow through a blood vessel, such as one or more renal veins 1208a, 1208b. The modulating and/or balancing of blood flow may be performed by device 1202 to occlude, partially occlude, and/or otherwise modulate or regulate blood flow to or through a portion of a blood vessel. In some examples, such management of blood flow to or through a blood vessel may result in additionally modulating pressure across one or both kidneys and/or other organs of the body. That is, the device 1202 may be used to manage blood flow to reduce central venous pressure. In particular, the device 1202 may be implanted in a blood vessel section between the IVC and a renal vein and may be actuated to reduce renal outlet pressure and thus improve ureter output and/or kidney function by increasing a gradient on the kidney.

In some examples, the flow modulating device 1202 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 1202 may be used as a method of treatment to regulate pressure in the right atrium of the heart. Further, the flow modulating device 1202 may be used as a method of treatment to improve function of the kidneys in patients having reduced kidney function due to pressure in the venous system.

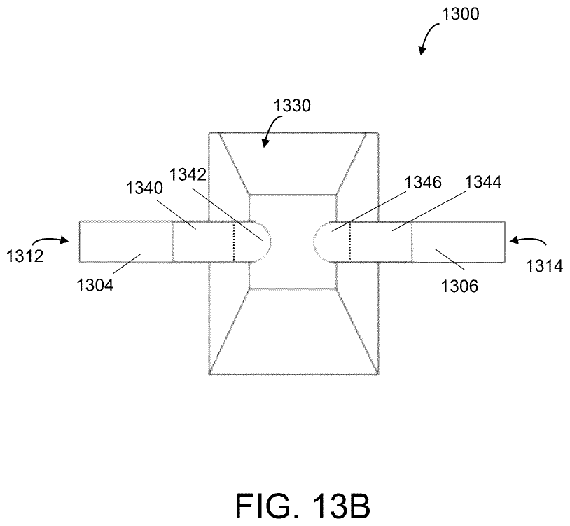

FIG. 13A illustrates a side view of an example flow modulating device 1300 for implantation into portions of an IVC, a left renal vein, and/or a right renal vein. The device 1300 includes a stent 1302 that is coupled to a first conduit 1304 and coupled to a second conduit 1306. The first conduit 1304 is coupled to stent 1302 through a first opening 1308. The first opening 1308 is shown as substantially round, but other shapes may be substituted including, but not limited to an oval, a triangle, a rectangle, a square, a polygon, etc. Similarly, the second conduit 1304 is coupled to the stent 1302 through a second opening 1310. In general, the shape of the conduits 1304, 1306 match a shape of the respective openings 1308, 1310 that receive the conduits 1304, 1306. The interface between the conduit 1304 and the opening 1308 is sealable to ensure that blood may flow from orifice 1312 into stent 1302 without leaking around opening 1308. Similarly, the interface between the conduit 1306 and the opening 1310 is sealable to ensure that blood may flow from orifice 1314 into stent 1302 without leaking around opening 1310. The stent 1300 may have a length between about 90 millimeters to about 220 millimeters. However, other sizes and/or shapes are also within the scope of this disclosure.

In some examples, conduit 1304 is not part of device 1300 and instead opening 1308 is sealed or not cut into stent 1302. For example, a left renal vessel may have the conduit 1306 implanted, but a right renal vessel may not have an implant therein. In some examples, neither conduit 1304 nor conduit 1306 may be implanted and instead a tube or other flow device may be installed within device 1300 to direct blood flow through a left renal vessel, a right renal vessel, and/or the IVC. The decision to implant zero, one or two conduits associated with device 1300 may be based on a diagnosis with respect to the heart, one or both renal vessels, the IVC, and/or one or both kidneys.

The stent 1300 is depicted in an hourglass-shaped form having five distinct portions along a longitudinal axis L. The stent 1300 includes an outflow end 1330 and an inflow end 1332 for flowing blood through device 1300. The first portion 1316 may have a diameter between about 20 millimeters and about 22 millimeters. The second portion 1318 may have a first diameter between about 20 millimeters and about 22 millimeters and may slope down to a second diameter that is between about 8 millimeters and about 10 millimeters. The third portion 1320 may have a diameter between about 8 millimeters and about 10 millimeters. The fourth portion 1322 may have a first diameter between about 8 millimeters and about 10 millimeters and may slope up to a second diameter that is between about 20 millimeters and about 22 millimeters. The fifth portion 1324 may have a diameter between about 20 millimeters and about 22 millimeters. While an hourglass stent with five portions is depicted in FIG. 13A, other shapes and/or more or fewer portions of an hourglass shape or other shapes and/or combination of shapes may be contemplated.

The conduits 1304, 1306 may have a length between about 30 millimeters to about 70 millimeters. In some examples, conduit 1304 is the same size and shape as conduit 1306. In some examples, conduit 1304 is about 10 percent to about 20 percent longer than conduit 1306. In some examples, conduit 1304 is about 10 percent to about 20 percent shorter than conduit 1306. The conduits 1304, 1306 are generally sized to be placed within a respective right, left renal artery. However, other sizes and/or shapes are also within the scope of this disclosure.

The stent 1300 may have a diameter between about 5 millimeters and about 20 millimeters. In some examples, cross-section of the stent frame 110 is oval in shape. Accordingly, the diameter of stent 1300 (e.g., associated with a major axis of the stent) may pertain to a largest distance across the cross-section of the oval (i.e., longest edge to edge measurement of the oval of the cross-section).

In operation, the device 1300 may function to funnel (e.g., narrow) portions of the renal artery, for example, to generate a Venturi effect (e.g., suction effect) within the IVC thereby accelerating blood flow velocity through the IVC as the cross-sectional area of the stent converges. Such a design may lead to a lower pressure at the outflow end 1330 due to the increased velocity of the blood through the device 1300.

The device 1300 may be configured to be delivered to a blood vessel in a compressed configuration. A stent wall of stent 1300 may be an open cell wall and/or may be adapted to be secured to a blood vessel wall of a blood vessel through endothelialization and/or using fasteners (e.g., one or more hooks, barbs, and/or other attachment features/means adapted to facilitate secure attachment of the stent 1300 to the tissue of the target blood vessel wall). When the device 1300 is implanted within the blood vessel, the stent wall may be configured to radially expand into substantially direct surface contact with the blood vessel wall (e.g., the wall of a renal vein).

In some examples, the device 1300 may be at least partially composed of a shape memory alloy, such as Nitinol®. In some examples, the device 1300 may be at least partially composed of cobalt-chrome. In some examples, the device 1300 may be at least partially composed of a polymer. In some examples, the device 1300 may be at least partially composed of a biodegradable material. In some examples, the device 1300 may be at least partially composed of any combination of Nitinol®, cobalt-chrome, a polymer, a metal, and/or a biodegradable material.

FIG. 13B illustrates a partial top down view of the example flow modulating device 1300 for implantation into portions of an IVC, a left renal vein, and/or a right renal vein. The partial view is sliced at line A shown in FIG. 13A to depict a portion of the conduit 1304 and conduit 1306. As shown, the conduit 1304 includes a stent frame 1340 (e.g., stent frame 508, stent frame 800, etc.) implanted therein. The stent frame 1340 includes a valve 1342 (e.g., valve 512, valve 812, etc.). The valve 1342 may operate similar to the valves described herein to modulate blood flow through a renal vein, when the device 1300 is implanted.

The conduit 1306 includes a stent frame 1344 (e.g., stent frame 508, stent frame 800, etc.) implanted therein. The stent frame 1344 includes a valve 1346 (e.g., valve 512, valve 812, etc.). The valve 1346 may operate similar to the valves described herein to modulate blood flow through a renal vein, when the device 1300 is implanted. The stent frame 1340 and stent frame 1344 may further function to modulate blood flow through the IVC when device 1300 is implanted.

As used herein, the term “active” with respect to blood flow management may represent operations carried out by the devices described herein using power or controller induced movement. For example, actively moving a portion of the devices described herein may include the use of battery power, wall outlet power, magnetic field induction, electromagnetic field induction, magnetic polarization, a piston-based system, a valve based system (e.g., with a manifold), hydraulics, pneumatics, optical actuators, thermal actuators, and/or other actuator using electrical or inductive power.

In some examples, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include an actuator (e.g., a linear actuator) coupled to a control element of the flow management device. The linear actuator tensions the control element to position the valve of the flow management device in a restricted blood flow state. Alternatively, the linear actuator releases tension in the control element to position a valve, a membrane, or other material in an unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote control device, or otherwise in wireless or electrical communication with a flow management system. The sensor can be a strain gauge, a piezoelectric sensor, a capacitance sensor, or a vacuum pressure sensor, such that the sensor senses a pressure in the blood vessel.

As used herein, the term “passive” with respect to blood flow management may represent operations carried out by the devices described herein using passively induced movement. For example, passively moving a portion of the devices described herein may include the use of manual pull wires (e.g., sutures, actuation wires/cords, etc.), anatomy responses (e.g., changes in vessel inner diameter, intra-vessel pressure, etc.), blood movement, or the like.

Any of the implantable or flow modulating devices described herein may be coated with a polymer (e.g., silicones, poly (urethanes), poly (acrylates), or copolymers such as poly (ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly(ethylene terephthalate), while the nonwoven fabrics may be made from expanded poly (tetrafluoroethylene). Some textiles may also or alternatively include silk, silk-based materials, or other textiles.

Further, any of actuation mechanisms described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol®, palladium, gold and their alloys, etc.), or a combination thereof.

The flow modulating devices described herein may be part of (or installed within) a stent. The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

The stents/stent frames described herein may be configured to house all or a portion of the flow modulating devices described herein. Such stents may include an assembly with strut members interconnected by joints that form a series of linked mechanisms that result in a hollow tube-shaped element. The stents may be positioned and/or repositioned within a blood vessel to introduce or remove flow modulating devices or device members including, but not limited, to valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device. For example, a stent or stent portion may surround a portion of a flow modulating device to ensure the device remains in a specified position in a blood vessel. In some examples, the stent surrounds the flow modulating device entirely. In some examples, the stent surrounds the flow modulating device and further continues beyond one or both ends of the device.

As used herein, the term “oval” may be used substantially interchangeably with the terms “ellipse” and/or “oblong.” The term “oval” may be used to refer to any non-circular closed curve having major and minor axes, the major axis being greater than the minor axis. With respect to “oval”-shaped stent frames disclosed herein, such stent frames may have relatively flatter minor-axis sidewalls (compared to curved major-axis end walls), where the sidewalls may bow radially outward, and/or may be deflected/curved radially inward so as to produce external concavity and internal convexity in such sidewalls.

The stents/stent frames described herein may include an outer frame. The outer frame may have a form and structure that varies. For example, the strut members and/or articulated joints may form a mesh-like structure. The strut members may be interconnected in such a way as to form a shaped pattern of cells. For example, any number of strut members may form a ring of the stent such that the strut members are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of strut members interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a renal procedure, a catheter tip and/or catheter may be configured to pass from the IVC into a renal vein to implant the flow modulating device. Further, for central venous access, a catheter tip and/or catheter may be configured to pass from the femoral vein into the IVC to implant the device into a portion of the renal vein and/or a portion of the IVC.

In some examples, the delivery system may include a trocar or other suitable delivery device used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., superior vena cava, inferior vena cava, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example delivery catheters, cannulas, and/or trocars. It will be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1. An implantable device for modulating blood flow through a blood vessel, the implantable device comprising: a stent frame positionable within the blood vessel, the stent frame having a substantially oval cross-sectional shape; and a valve defining an inflow end and an outflow end, the inflow end being coupled to the stent frame and the outflow end defining an aperture, the valve comprising at least one leaflet at the outflow end, the at least one leaflet being positionable to overlay at least a portion of the aperture and configured to move in a radial direction that is perpendicular to a central axis of the stent frame, wherein the valve is configured to cause the at least one leaflet to move in the radial direction to reduce a cross-sectional area of the aperture and restrict flow through the valve in response to elevated pressure within the blood vessel.

Example 2. The example of any of the preceding examples, but particularly example 1, wherein, wherein: the blood vessel is a renal vein leading to a kidney; and the at least one leaflet is configured to reduce a back flow of blood toward the kidney from the renal vein by at least partially obstructing the aperture in response to detecting a pressure within an inferior vena cava, fluidly connected to a portion of the renal vein, that is greater than a predefined pressure threshold.

Example 3. The example of any of the preceding examples, but particularly the implantable device of example 1, wherein: the blood vessel is a renal vein leading to a kidney; and the at least one leaflet is configured to reduce a back flow of blood toward the kidney from the renal vein by at least partially obstructing the aperture in response to detecting a pressure within the renal vein that is greater than a predefined pressure threshold.

Example 4. The example of any of the preceding examples, but particularly the implantable device of example 1, further comprising at least one control element coupled to the valve, wherein the at least one control element is configured to move the at least one leaflet between a first position and a second position to manipulate the valve between an unrestricted blood flow position and a restricted blood flow position.

Example 5. The example of any of the preceding examples, but particularly the implantable device of example 4, wherein: the unrestricted blood flow position comprises the at least one leaflet moving radially away from the central axis of the stent frame to cause an increase in blood flow through the valve; and the restricted blood flow position comprises the at least one leaflet moving radially toward the central axis of the stent frame to cause a decrease in blood flow through the valve.

Example 6. The example of any of the preceding examples, but particularly the implantable device of example 4, wherein: the first position comprises the at least one leaflet at least partially obstructing the aperture; and the second position comprises the at least one leaflet being arranged so that the aperture is substantially unobstructed.

Example 7. The example of any of the preceding examples, but particularly the implantable device of example 1, wherein the valve is a one-way valve configured to modulate blood flow through the valve and from at least one organ.

Example 8. The example of any of the preceding examples, but particularly the implantable device of example 1, wherein the valve further comprises one or more additional leaflets arranged with the at least one leaflet, wherein the at least one leaflet and the one or more additional leaflets are configured to proportionally move toward the central axis of the stent frame in response to the elevated pressure within the blood vessel to restrict flow through the valve.

Example 9. The example of any of the preceding examples, but particularly the implantable device of example 1, wherein a diameter of the aperture is about 5 millimeters to about 20 millimeters.

Example 10. The example of any of the preceding examples, but particularly the implantable device of example 1, wherein: the blood vessel comprises a renal vein extending from a kidney to an inferior vena cava; and the elevated pressure is associated with the inferior vena cava.

Example 11. The example of any of the preceding examples, but particularly the implantable device of example 1, wherein: the stent frame is configured to pull a wall of the blood vessel toward the central axis to modify a portion of the blood vessel from a substantially circular cross-sectional shape to a substantially oval cross-sectional shape; and the aperture has a substantially oval cross-sectional shape.

Example 12. The example of any of the preceding examples, but particularly the implantable device of example 1, further comprising at least one elastomeric member having a first opening and a second opening and defining a first lumen, the first opening being coupled to a portion of the stent frame, wherein the first lumen defines: a first cross-sectional area at the first opening that extends for a first portion of a length of the member, a second cross-sectional area that extends over a second portion of the length of the member, and a third cross-sectional area that extends over a third portion of the length of the member, the first cross-sectional area being greater than the second cross-sectional area, the second cross-sectional area being greater than the third cross-sectional area, wherein the at least one elastomeric member is configured to accelerate a flow velocity of blood through the first lumen and lower a flow pressure associated with the blood vessel.

Example 13. The example of any of the preceding examples, but particularly the implantable device of example 12, further comprising an additional elastomeric member having a third opening and a fourth opening and defining a second lumen, the third opening being coupled to a second portion of the stent frame, wherein the second lumen defines a fourth cross-sectional area at the third opening that extends for a first portion of a length of the additional member, a fifth cross-sectional area for a second portion of the length of the additional member, a sixth cross-sectional area for a third portion of the length of the additional member, the fourth cross-sectional area being greater than the fifth cross-sectional area, the fifth cross-sectional area being greater than the sixth cross-sectional area, wherein the additional elastomeric member is configured to accelerate a flow velocity of blood through the second lumen and lower the flow pressure associated with the blood vessel.

Example 14. The example of any of the preceding examples, but particularly the implantable device of example 1, wherein the valve comprises or is formed of one or more of: a polymer, a biomaterial, or a textile.

Example 15. The example of any of the preceding examples, but particularly the implantable device of example 1, further comprising: at least one sensor coupled to the implantable device, the sensor being configured to detect the elevated pressure; a processor coupled to the at least one sensor, and a control element coupled to the valve, the at least one sensor, and the processor wherein the at least one control element is configured to move the at least one leaflet to an unrestricted blood flow position when the at least one sensor detects a blood vessel pressure below a predefined pressure level and to move the at least one leaflet to a restricted blood flow position when the at least one sensor detects the blood vessel pressure is at or above the predefined pressure level.

Example 16. An implantable device for modulating blood flow through a blood vessel, the device comprising: a stent frame positionable within the blood vessel, the stent frame having a substantially oval cross-sectional shape; a valve defining an inflow end and an outflow end defining an aperture, the valve comprising at least one leaflet at the outflow end, the at least one leaflet being positionable to overlay at least a portion of the aperture and configured to move in a radial direction that is perpendicular to a central axis of the stent frame; a first elastomeric member having a first opening and a second opening and defining a first lumen, the first opening being coupled to a portion of the stent frame, the second opening being coupled to a portion of the inflow end of the valve; and a second elastomeric member having a third opening and a fourth opening and defining a second lumen, the third opening being coupled to a portion of the stent frame, the fourth opening being coupled to a portion of the inflow end of the valve, wherein the valve is configured to cause the at least one leaflet to move in the radial direction to reduce a cross-sectional area of the aperture and restrict flow through the valve in response to elevated pressure within the blood vessel.

Example 17. The example of any of the preceding examples, but particularly the implantable device of example 16, wherein: the implantable device is implanted in a renal vein; the valve is positioned within an inferior vena cava; and the at least one leaflet is configured to reduce a back flow of blood toward a kidney and from the renal vein by at least partially obstructing the aperture in response to the elevated pressure, wherein the elevated pressure is within the inferior vena cava.

Example 18. The example of any of the preceding examples, but particularly the implantable device of example 16, wherein: the blood vessel is a renal vein leading to a kidney; and the at least one leaflet is configured to reduce a back flow of blood toward the kidney from the renal vein by at least partially obstructing the aperture in response to detecting a pressure within the renal vein that is greater than a predefined pressure threshold.

Example 19. The example of any of the preceding examples, but particularly the implantable device of example 16, further comprising at least one control element coupled to the valve, wherein the at least one control element is configured to move the at least one leaflet between a first position and a second position to manipulate the valve between an unrestricted blood flow position and a restricted blood flow position.

Example 20. The example of any of the preceding examples, but particularly the implantable device of example 19, wherein: the unrestricted blood flow position comprises the at least one leaflet moving radially away from the central axis of the stent frame to cause an increase in blood flow through the valve; and the restricted blood flow position comprises the at least one leaflet moving radially toward the central axis of the stent frame to cause a decrease in blood flow through the valve.

Example 21. The example of any of the preceding examples, but particularly the implantable device of example 19, wherein: the first position comprises the at least one leaflet at least partially obstructing the aperture; and the second position comprises the at least one leaflet being arranged so that the aperture is substantially unobstructed.

Example 22. The example of any of the preceding examples, but particularly the implantable device of example 16, wherein the valve further comprises one or more additional leaflets arranged with the at least one leaflet, wherein the at least one leaflet and the one or more additional leaflets are configured to proportionally move toward the central axis of the stent frame in response to the elevated pressure within the blood vessel to restrict flow through the valve.

Example 23. The example of any of the preceding examples, but particularly the implantable device of example 16, wherein a diameter of the aperture is about 5 millimeters to about 20 millimeters.

Example 24. The example of any of the preceding examples, but particularly the implantable device of example 16, wherein the valve comprises or is formed of one or more of: a polymer, a biomaterial, or a textile.

Example 25. The example of any of the preceding examples, but particularly the implantable device of example 16, wherein: the stent frame is configured to pull a wall of the blood vessel toward the central axis to modify a portion of the blood vessel from a substantially circular cross-sectional shape to a substantially oval cross-sectional shape; and the aperture has a substantially oval cross-sectional shape.

Example 26. A method of modulating a pressure gradient between a first kidney and a second kidney of a subject, the method comprising: introducing an implantable device in a renal vein, the device comprising: a stent frame with an oval-shaped cross-sectional area; and a valve defining an inflow end and an outflow end, the inflow end being coupled to the stent frame and the outflow end defining an aperture, the valve comprising at least one leaflet at the outflow end of the valve, the at least one leaflet being positionable to overlay at least a portion of the aperture and configured to move in a radial direction that is perpendicular to a central axis of the stent frame; and actuating the implantable device to modulate a flow of blood within the renal vein, the actuating comprising causing the at least one leaflet to move in the radial direction to increase or decrease a cross-sectional area of the aperture.

Example 27. The method of any of the preceding examples, but particularly the method of example 26, wherein increasing the cross-sectional area of the aperture results in unrestricting flow through the valve.

Example 28. The method of any of the preceding examples, but particularly the method of example 26, wherein decreasing the cross-sectional area of the aperture results in restricting flow through the valve.

Example 29. The method of any of the preceding examples, but particularly the method of example 26, wherein the valve further comprises one or more additional leaflets arranged with the at least one leaflet, wherein the at least one leaflet and the one or more additional leaflets are configured to move toward the central axis of the stent frame to restrict flow through the valve when there is an elevated pressure within the renal vein.

Example 30. The method of any of the preceding examples, but particularly the method of example 26, wherein a diameter of the aperture is about 5 millimeters to about 20 millimeters.

Example 31. The method of any of the preceding examples, but particularly the method of example 26, wherein the valve comprises or is formed of one or more of: a polymer, a biomaterial, or a textile.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the control device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “projection” may include, and is contemplated to include, a plurality of projections. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An implantable device for modulating blood flow through a blood vessel, the implantable device comprising:
a stent frame positionable within the blood vessel, the stent frame having a substantially oval cross-sectional shape;
a valve defining an inflow end and an outflow end, the inflow end being coupled to the stent frame and the outflow end defining an aperture, the valve comprising at least one leaflet at the outflow end, the at least one leaflet being positionable to overlay at least a portion of the aperture and configured to move in a radial direction; and
at least one control element coupled to the valve,
wherein the at least one control element is configured to cause the valve to move the at least one leaflet in the radial direction between a first position and a second position to restrict flow through the valve in response to elevated pressure within the blood vessel.

2. The implantable device of claim 1, wherein:
the blood vessel is a renal vein leading to a kidney; and
the at least one leaflet is configured to reduce a back flow of blood toward the kidney from the renal vein by at least partially obstructing the aperture in response to detecting a pressure within an inferior vena cava, fluidly connected to a portion of the renal vein, that is greater than a predefined pressure threshold.

3. The implantable device of claim 1, wherein:
the blood vessel is a renal vein leading to a kidney; and
the at least one leaflet is configured to reduce a back flow of blood toward the kidney from the renal vein by at least partially obstructing the aperture in response to detecting a pressure within the renal vein that is greater than a predefined pressure threshold.

4. The implantable device of claim 1, wherein:
the first position is an is an unrestricted blood flow position comprising the at least one leaflet moving radially away from a central axis of the stent frame to cause an increase in blood flow through the valve; and
the second position is a restricted blood flow position comprising the at least one leaflet moving radially toward the central axis of the stent frame to cause a decrease in blood flow through the valve.

5. The implantable device of claim 1, wherein:
the first position comprises the at least one leaflet at least partially obstructing the aperture; and
the second position comprises the at least one leaflet being arranged so that the aperture is substantially unobstructed.

6. The implantable device of claim 1, wherein the valve further comprises one or more additional leaflets arranged with the at least one leaflet, wherein the at least one leaflet and the one or more additional leaflets are configured to proportionally move toward a central axis of the stent frame in response to the elevated pressure within the blood vessel to restrict flow through the valve.

7. The implantable device of claim 1, wherein:
the stent frame is configured to pull a wall of the blood vessel toward a central axis to modify a portion of the blood vessel from a substantially circular cross-sectional shape to a substantially oval cross-sectional shape; and
the aperture has a substantially oval cross-sectional shape.

8. The implantable device of claim 1, further comprising at least one elastomeric member having a first opening and a second opening and defining a first lumen, the first opening being coupled to a portion of the stent frame,
wherein the first lumen defines:
a first cross-sectional area at the first opening that extends for a first portion of a length of the member,
a second cross-sectional area that extends over a second portion of the length of the member, and
a third cross-sectional area that extends over a third portion of the length of the member, the first cross-sectional area being greater than the second cross-sectional area, the second cross-sectional area being greater than the third cross-sectional area,
wherein the at least one elastomeric member is configured to accelerate a flow velocity of blood through the first lumen and lower a flow pressure associated with the blood vessel.

9. The implantable device of claim 8, further comprising an additional elastomeric member having a third opening and a fourth opening and defining a second lumen, the third opening being coupled to a second portion of the stent frame,
wherein the second lumen defines a fourth cross-sectional area at the third opening that extends for a first portion of a length of the additional member, a fifth cross-sectional area for a second portion of the length of the additional member, a sixth cross-sectional area for a third portion of the length of the additional member, the fourth cross-sectional area being greater than the fifth cross-sectional area, the fifth cross-sectional area being greater than the sixth cross-sectional area,
wherein the additional elastomeric member is configured to accelerate a flow velocity of blood through the second lumen and lower the flow pressure associated with the blood vessel.

10. The implantable device of claim 1, further comprising:
at least one sensor coupled to the implantable device, the sensor being configured to detect the elevated pressure;
a processor coupled to the at least one sensor, and
a control element coupled to the valve, the at least one sensor, and the processor
wherein the control element is configured to move the at least one leaflet to an unrestricted blood flow position when the at least one sensor detects a blood vessel pressure below a predefined pressure level and to move the at least one leaflet to a restricted blood flow position when the at least one sensor detects the blood vessel pressure is at or above the predefined pressure level.

11. An implantable device for modulating blood flow through a blood vessel, the device comprising:
a stent frame positionable within the blood vessel, the stent frame having a substantially oval cross-sectional shape;
a valve defining an inflow end and an outflow end defining an aperture, the valve comprising at least one leaflet at the outflow end, the at least one leaflet being positionable to overlay at least a portion of the aperture and configured to move in a radial direction that is perpendicular to a central axis of the stent frame;
a first elastomeric member having a first opening and a second opening and defining a first lumen, the first opening being coupled to a portion of the stent frame, the second opening being coupled to a portion of the inflow end of the valve; and
a second elastomeric member having a third opening and a fourth opening and defining a second lumen, the third opening being coupled to a portion of the stent frame, the fourth opening being coupled to a portion of the inflow end of the valve,
wherein the valve is configured to cause the at least one leaflet to move in the radial direction to reduce a cross-sectional area of the aperture and restrict flow through the valve in response to elevated pressure within the blood vessel.

12. The implantable device of claim 11, wherein:
the blood vessel is a renal vein leading to a kidney; and
the at least one leaflet is configured to reduce a back flow of blood toward the kidney from the renal vein by at least partially obstructing the aperture in response to detecting a pressure within the renal vein that is greater than a predefined pressure threshold.

13. The implantable device of claim 11, further comprising at least one control element coupled to the valve, wherein the at least one control element is configured to move the at least one leaflet between a first position and a second position to manipulate the valve between an unrestricted blood flow position and a restricted blood flow position.

14. The implantable device of claim 13, wherein:
the unrestricted blood flow position comprises the at least one leaflet moving radially away from the central axis of the stent frame to cause an increase in blood flow through the valve; and
the restricted blood flow position comprises the at least one leaflet moving radially toward the central axis of the stent frame to cause a decrease in blood flow through the valve.

15. The implantable device of claim 13, wherein:
the first position comprises the at least one leaflet at least partially obstructing the aperture; and
the second position comprises the at least one leaflet being arranged so that the aperture is substantially unobstructed.

16. The implantable device of claim 11, wherein the valve further comprises one or more additional leaflets arranged with the at least one leaflet, wherein the at least one leaflet and the one or more additional leaflets are configured to proportionally move toward the central axis of the stent frame in response to the elevated pressure within the blood vessel to restrict flow through the valve.

17. The implantable device of claim 11, wherein:
the stent frame is configured to pull a wall of the blood vessel toward the central axis to modify a portion of the blood vessel from a substantially circular cross-sectional shape to a substantially oval cross-sectional shape; and
the aperture has a substantially oval cross-sectional shape.

18. An implantable device for modulating blood flow through a blood vessel, the implantable device comprising:
a stent frame positionable within the blood vessel, the stent frame having a substantially oval cross-sectional shape;
a valve defining an inflow end and an outflow end, the inflow end being coupled to the stent frame and the outflow end defining an aperture, the valve comprising at least one leaflet at the outflow end, the at least one leaflet being positionable to selectively cover at least a portion of the aperture; and
at least one control element coupled to the valve,
wherein the at least one control element is configured to move the at least one leaflet between a first position and a second position to manipulate the valve between a substantially unrestricted blood flow position and a restricted blood flow position,
wherein the at least one leaflet at least partially obstructs the aperture in the first position and wherein the aperture is substantially unobstructed in the second position.

19. The implantable device of claim 18, wherein the blood vessel is a renal vein leading to a kidney, and wherein the at least one leaflet is configured to reduce a back flow of blood toward the kidney from the renal vein in response to detecting a pressure in an inferior vena cava.

20. The implantable device of claim 18, wherein the blood vessel is a coronary sinus, and wherein the at least one leaflet is provided for selectively obstructing the aperture and thereby regulating blood flow through the coronary sinus.
